(19) United States
(12) Patent Application Publication
(10) Pub. No.: US 2026/0363960 A1
(43) Pub. Date: Nov. 19, 2026

(54) INTELLIGENT MEDICATION ADHERENCE SYSTEM WITH PERSONALIZED HEALTH-ADAPTIVE DISPENSING

(71) Applicant: Smartmed Devices and Services Corporation, Fremont, CA (US)

(72) Inventor: Abdul Raheem Khan, Fremont, CA (US)

(73) Assignee: Smartmed Devices and Services Corporation, Fremont, CA (US)

(21) Appl. No.: 19/677,906

(22) Filed: May 14, 2026

Related U.S. Application Data
(60) Provisional application No. 63/806,095, filed on May 15, 2025.

(51) Int. Cl. A61J 7/04; G16H 20/13

(52) U.S. Cl. CPC A61J 7/049; G16H 20/13; A61J 2200/30; A61J 2200/74; A61J 2205/30

(57) ABSTRACT

A method, system, and computer-readable storage media for operating a medication dispensing apparatus are disclosed. One or more processors obtain a user profile record, a device identifier of a peripheral data source paired with a user, a structured prescription record for a medication, and a placement record indicating an assignment of a medication container to a designated compartment of the medication dispensing apparatus. A composite identifier is derived from the obtained records. A control instruction addressing the designated compartment is generated, the control instruction being conditioned on a correspondence between the placement record and the designated compartment. The control instruction is transmitted through a communications interface to the medication dispensing apparatus to cause a physical state change of the designated compartment. An event record indicating an outcome of the physical state change is recorded and linked to the composite identifier.

### PRIORITY APPLICATION

This application claims the benefit of U.S. Provisional Patent Application No. 63/806,095, filed on May 15, 2025, titled “Smart Medication Adherence System” (Atty. Docket No. SDSC1000USP01) which is hereby incorporated by reference for all purposes.

### TECHNICAL FIELD

The technology disclosed relates to artificial intelligence type computers and digital data processing systems and corresponding data processing methods and products for emulation of intelligence (i.e., knowledge based systems, reasoning systems, and knowledge acquisition systems); and including systems for reasoning with uncertainty (e.g., fuzzy logic systems), adaptive systems, machine learning systems, and artificial neural networks. Specifically, the present disclosure relates generally to medication management systems and methods. More particularly, the present disclosure relates to systems, methods, and computer-readable storage media for operating a medication dispensing apparatus.

### BACKGROUND

Medication adherence is a foundational component of effective healthcare. The therapeutic outcome of a prescription may depend on a sequence of operations that may include prescription validation, scheduled dispensing, identification of medication and recipient, measurement of dispensed quantity, and recording of the dispensing event in a form that downstream stakeholders may rely on. The patient population that may benefit from accurate adherence support may include individuals managing chronic conditions, individuals recovering from surgical procedures, individuals on complex polypharmacy regimens, and individuals in supervised care environments such as assisted-living facilities, nursing homes, hospitals, and clinical trial centers.

Conventional approaches to medication management may include various tools, applications, and devices that each may address one or more aspects of the dispensing process. Audit records and operational data produced by such conventional approaches may originate from multiple independent subsystems. Improvements in medication management technology that may address one or more of these aspects may therefore be beneficial.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 illustrates a system architecture of a medication adherence and personalized health management platform for operating a medication dispensing apparatus, according to certain embodiments.

FIG. 2 illustrates a composite identifier generation and operational module pipeline of a medication adherence and personalized health management system, according to certain embodiments.

FIG. 3A illustrates a medication dispensing apparatus having a carousel-based dispensing architecture and example compartment configurations, in accordance with an embodiment.

FIG. 3B illustrates an alternative node-based medication storage unit that may implement one or more compartments of the medication dispensing apparatus, in accordance with an embodiment.

FIG. 4 is a block diagram of an electrical and computational architecture of a medication dispensing apparatus, according to certain embodiments.

FIG. 5 illustrates a prescription extraction pipeline of a medication adherence and personalized health management system for extracting a structured prescription record from a multi-angle image sequence of a prescription label, according to certain embodiments.

FIG. 6 is a flowchart of a method of operating a medication dispensing apparatus including deriving a composite identifier and causing a physical state change of a designated compartment, according to certain embodiments.

FIG. 7 is a flowchart of a method of generating a verification result for a dispensing operation through weight-based consumption verification with a tiered fallback initial-units quantity determination, according to certain embodiments.

FIG. 8A illustrates a wellness score feedback loop of a medication adherence and personalized health management system for computing a wellness score from a plurality of biometric signals, according to certain embodiments.

FIG. 8B illustrates a temporal escalation and recipient hierarchy pipeline of a medication adherence and personalized health management system, according to certain embodiments.

FIG. 8C illustrates an event-record association, protected-storage, and audit-access workflow, in accordance with an embodiment.

FIG. 9 is a schematic representation of an encoder-decoder architecture.

FIG. 10 shows an overview of an attention mechanism added onto an RNN encoder-decoder architecture.

FIG. 11 is a schematic representation of the calculation of self-attention showing one attention head.

FIG. 12 is a depiction of several attention heads in a Transformer block.

FIG. 13 is an illustration that shows how one can use multiple workers to compute the multi-head attention in parallel, as the respective heads compute their outputs independently of one another.

FIG. 14 is a portrayal of one encoder layer of a Transformer network.

FIG. 15 shows a schematic overview of a Transformer model.

FIG. 16 is a depiction of a Vision Transformer (ViT).

FIG. 17 illustrates a processing flow of the Vision Transformer (ViT).

FIG. 18 shows example software code that implements a Transformer block.

FIG. 19 illustrates an example configuration of a computing device that can be used to implement the systems and techniques described herein.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

### Overview

The implementations described herein introduce systems and methods for operating a medication dispensing apparatus through an integrated computational pipeline that ties prescription acquisition, identity construction, contextual health scoring, sensor-based consumption verification, and physical dispensing control into a single coordinated framework. In some implementations, one or more processors derive a composite identifier (CI) from a user profile record, a device identifier of a peripheral data source paired with a user, a structured prescription record for a medication associated with a medication container, and a placement record indicating an assignment of the medication container to a designated compartment of the medication dispensing apparatus. The one or more processors generate a control instruction that addresses the designated compartment, transmit the control instruction through a communications interface to the medication dispensing apparatus to cause a physical state change of the designated compartment, and record an event record linked to the CI on a non-transitory storage medium. The system applies a trained inference model to image data of a prescription label to extract the structured prescription record and validates the structured prescription record against a pharmacological reference data store. The system records the event record in association with the composite identifier and may store the event record using secure storage, access-control, synchronization, or other data-protection techniques. The system also receives weight measurements from a weight sensor of the medication dispensing apparatus, computes a consumed-units quantity from a weight differential, generates a verification result, and modifies a subsequent dispensing operation when the verification result indicates a discrepancy exceeding a configurable tolerance value.

The disclosed approach treats the CI as a primary key for every event associated with the medication of the user, enabling deterministic coordination across the prescription, biometric, dispensing, and audit functions that conventional systems treat as separate workflows. The system achieves real-time closed-loop control between a wellness score derived from biometric signals and the control instruction transmitted to the medication dispensing apparatus, eliminating the disconnection between observation and action that limits conventional smart pill bottles and reminder applications.

### Indicia of Novelty, Inventiveness, Non-Obviousness, and Subject-Matter Eligibility

Across the world, healthcare depends on a fragile assumption. After a doctor writes a prescription and a pharmacist fills it, the medication leaves the system and the responsibility for taking it correctly falls on the patient. The medication that was carefully prescribed, carefully tested, and carefully manufactured must now be taken at the right time, in the right amount, by the right person, day after day, often for years. The assumption holds when the patient is young, healthy, attentive, and managing a single medication. The assumption breaks under the conditions in which most chronic care actually happens. Elderly patients juggle seven or eight prescriptions at once. Post-surgical patients follow complex tapering schedules. Dementia patients forget what they took an hour ago. Parents manage their own medications while caring for children. Working adults lead busy lives and let a dose slip through the cracks.

The cost of the fragile assumption is enormous. Medication non-adherence contributes to roughly 125,000 preventable deaths in the United States every year and accounts for hundreds of billions of dollars in avoidable hospitalizations, repeated prescriptions, and emergency interventions. Hospitals lose money to readmissions caused by missed doses. Insurance companies pay for complications that better adherence would have avoided. Families lose loved ones to events that, on paper, were entirely preventable. The healthcare system has known about the problem for decades and has tried to address it from many directions, but the problem persists at scale because the tools used to address it are limited in a fundamental way.

The tools available today address only fragments of the medication delivery process, and each tool operates in its own small silo. A reminder application can send a notification, but it cannot verify whether the patient actually took the pill. A smart pill bottle can count weight changes, but it cannot confirm who took the medication or whether the bottle held the right medication for the right person. A pharmacy system can validate a prescription, but it cannot observe what happens after the bottle leaves the counter. A wearable device can stream heart rate and activity to a dashboard, but it cannot influence whether the next dose should proceed. The patient is left to integrate these tools personally, and the patient is the person most likely to forget. The caregiver, the doctor, and the compliance auditor are left to reconstruct what happened from incomplete records held in disconnected systems. When something goes wrong, no one has a complete picture, and no one can prove what was supposed to happen, what actually happened, or when the gap between the two became dangerous.

The technology disclosed addresses the problem by introducing a single coordinated system that supervises the entire medication delivery process from end to end. The system is not a new reminder, a smarter pill bottle, or a better wearable. The system is a coordinated framework that ties together every piece of information needed to confirm that the right person took the right medication at the right time in the right amount, and produces a single trustworthy record of what happened.

At the core of the technology disclosed is a unified record that combines four pieces of information: who the patient is, what device tracks the patient's health, what medication has been prescribed, and which physical compartment of the medication dispensing apparatus holds that medication. The unified record follows the medication and the patient through every step of the delivery process. When the system reads the prescription, the unified record is created. When the system decides whether to dispense a dose, the unified record is referenced. When the system records the outcome of the dispensing event, the unified record is the anchor. Every action carries the same identity, so every action can be traced, verified, and connected to every other action in a way that no fragmented system can match.

The setup is straightforward for the user. After unboxing the medication dispensing apparatus, the user installs a companion application on a smartphone and pairs the application with the dispensing apparatus and with a wearable device that the user already wears. When the user introduces a new medication, the user holds the prescription bottle in front of the camera of the smartphone or the dispensing apparatus. The system reads the prescription label using artificial intelligence trained model to recognize both printed and handwritten prescriptions, extracts the medication name, dosage, frequency, duration, and other fields, and verifies the extracted information against a comprehensive medical database to flag any drug interactions or contraindications. The system then asks the user to assign the bottle to a specific compartment in the dispensing apparatus. From that moment forward, the system knows precisely which medication is where, what schedule it follows, what risks it carries, and who is supposed to take it.

When the time for a dose arrives, the system performs a safety check before any medication is released. The system verifies that the bottle assigned to the compartment is the bottle that the system expects to be there. If anything is out of place, for example if the user has moved the bottle to a different compartment, or if a different bottle has been placed in the compartment by mistake, the system refuses to release the medication and records the exception. The safety check eliminates an entire class of medication errors that conventional tools cannot prevent because conventional tools do not check whether the right bottle is in the right slot before releasing the dose.

The technology disclosed also addresses several specific real-world risks that conventional medication management cannot manage in a coordinated way. For controlled substances including opioids, where uncontrolled access can lead to abuse, accidental overdose, or diversion, the system maintains every medication in a physically locked compartment that opens only at the scheduled time, only after the safety check passes, and only for the authorized patient. Accidental access by household members, including children who may mistake a medication for candy or for adult medications left within reach, is prevented because each compartment remains physically locked between scheduled dispensing events and is opened only in response to a verified control instruction. When a prescription changes and the patient loads a new bottle, the system associates the new bottle with the updated structured prescription record and discontinues the placement record for the previous bottle, preventing the accidental ingestion of an expired or discontinued medication from a bottle that the patient may have forgotten to discard. The system also tracks the remaining-units quantity for each medication in real time and projects the date on which the medication will run out based on the prescribed dosing schedule, allowing refill orders to be placed proactively rather than reactively. The abrupt discontinuation of treatment that frequently causes complications in chronic care, hypertension management, diabetes management, and other long-term therapeutic regimens is avoided because the system observes the consumption pattern and the remaining supply at every dispensing event and alerts the patient, the caregiver, the prescribing physician, and the pharmacy in advance of an anticipated run-out.

The system can compute the consumption accurately even when the prescription record does not specify the number of pills in the original bottle. The system first looks for an initial-count field in the prescription record. If the field is missing, the system consults a reference database of common pharmacy bottle weights and computes the count from the difference between the loaded bottle weight and the expected empty weight. If neither source applies, the system falls back to a sensible default. The three-step approach allows the system to operate reliably with prescriptions from any pharmacy, regardless of how complete the prescription metadata is.

The technology disclosed does not stop at verifying the dispensing event. The system also pays attention to the patient's body. Through the wearable device, the system continuously receives signals including heart rate, sleep patterns, physical activity, and environmental conditions. The system combines those signals with the patient's recent medication history, time of day, baseline health, and personal health profile to produce a wellness score that reflects the patient's current state. The wellness score is not a number that sits on a screen. The score informs the next dispensing decision. The schedule responds to the patient. The schedule does not run on autopilot.

If the patient misses a dose, the system does not simply send a reminder and then go quiet. The system progresses through a sequence of escalating alerts. The first alert is gentle. The second alert is more pronounced. The third alert is urgent. If the patient still does not respond, the system reaches out to a caregiver, a healthcare provider, or an emergency contact, in an order that the patient has configured during setup. The patient who lives alone is not alone. The patient who forgets is not forgotten. Help arrives before a missed dose becomes a hospitalization.

The result of all of these mechanisms is a single coordinated system that knows who is taking what, when, why, and whether the right thing actually happened. The patient sees a simple interface that confirms each dispensing event. The caregiver sees a real-time view of adherence and is notified when something goes wrong. The doctor receives reliable evidence of how the patient is actually following the regimen, not a self-report given at the next appointment three months later. The pharmacist can verify that the prescriptions are being taken as filled. The compliance officer at a regulated facility can produce a complete audit trail that withstands inspection.

### Technical Advantages

The technology disclosed offers a number of concrete and measurable technical advantages over the conventional approach.

The technology disclosed closes the verification gap at the dispensing event itself. Conventional smart pill bottles count weight changes, but the weight changes alone do not tell the system whether the right medication was removed, by the right user, at the right time. The unified record approach of the technology disclosed binds the weight measurement to the identity of the user, the identity of the medication, and the identity of the compartment, so the consumption check is meaningful at the moment it occurs rather than re-constructable only after the fact.

The technology disclosed prevents wrong-compartment dispensing through a safety check that operates at the level of the control instruction. Before the dispensing apparatus releases a dose, the system verifies that the bottle is in the compartment the prescription expects, and the system refuses to dispense when the verification fails. Conventional approaches that rely on the user to keep the bottles organized cannot prevent wrong-compartment errors, because conventional approaches make the dispensing decision without checking the placement.

The technology disclosed handles real-world prescription heterogeneity through a three-step fallback for determining the original pill count. Prescriptions from different pharmacies arrive with different levels of metadata. Some include the original pill count. Some do not. Some include unusual bottle sizes. The technology disclosed handles all of these cases automatically without requiring the user to perform manual calibration each time a new bottle is loaded.

The trained inference model that reads the prescription label handles both printed and handwritten prescriptions through a single integrated pipeline. Many medications are still prescribed on handwritten pads, and the technology disclosed does not require the user to type the prescription manually or to use a special pharmacy that supplies machine-readable labels. The user simply holds the bottle in front of the camera and the system handles the rest.

The technology disclosed adapts automatically to dispensing apparatuses with different compartment counts. The dispensing apparatus communicates the installed compartment count to the system at power-on through a configuration-encoding interface, and the system sets the indexing logic accordingly. The same software can operate a four-compartment, a six-compartment, or an eight-compartment apparatus without manual reconfiguration.

The technology disclosed coordinates the dispensing event, the verification result, and the escalation response through the same communications interface and the same unified record, producing a single source of truth that downstream caregivers, healthcare providers, regulators, and insurance companies can rely on without manual reconciliation. The audit overhead that consumes so much time in regulated environments is reduced because the audit trail is produced as a by-product of the dispensing operation rather than as a separate after-the-fact reconstruction.

For a healthcare system facing an aging population, a polypharmacy crisis, growing regulatory compliance burdens, and a chronic-disease epidemic that costs trillions over a generation, the technology disclosed is more than a product improvement. The technology disclosed represents a structural advance in how medication delivery is monitored, verified, and recorded. The dispensing apparatus is no longer a passive container for pills. The dispensing apparatus is an active participant in the patient's care, observing what happens at the point of delivery, deciding what should happen next based on the patient's current health state, acting on that decision through physical hardware, and producing a record that doctors, caregivers, regulators, and insurers can rely on.

For the millions of patients, families, and clinicians who carry the daily burden of medication management, the technology disclosed transforms an unverified handoff into a closed-loop process. The dose that is scheduled is the dose that is released. The dose that is released is the dose that is recorded against a real measurement of consumption. The record that is produced is the same record that every stakeholder sees. The difference between what the prescription said and what the patient actually did is no longer a question to be answered three months later at the next appointment. The difference is observed, measured, and addressed at the moment it matters. For the elderly patient living alone, for the post-surgical patient navigating a complex recovery, for the caregiver who calls every morning to ask whether mother remembered her heart medication, the technology disclosed is the difference between hoping the right thing happened and knowing the right thing happened.

### System Architecture

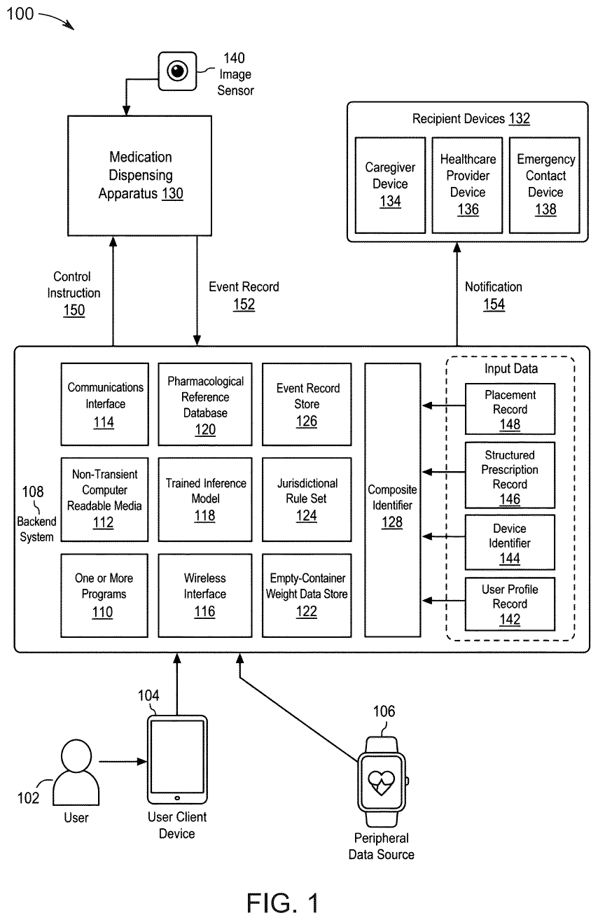

FIG. 1 illustrates a system 100 for operating a medication dispensing apparatus 130 and for managing medication adherence and personalized health for a user 102, in accordance with an embodiment. The system 100 may include the user 102, a user client device 104, a peripheral data source 106, a backend system 108, an image sensor 140, the medication dispensing apparatus 130, and one or more recipient devices 132. The system 100 coordinates prescription acquisition, user/device association, medication placement, controlled dispensing, sensor feedback, event recording, and health-context monitoring within a unified medication adherence workflow.

The user 102 may interact with the system 100 through the user client device 104, through a display or input component of the medication dispensing apparatus 130, through biometric or voice confirmation, or through caregiver-assisted input. The user client device 104 may include a smartphone, tablet, computer, wearable computing device, or other user-facing device configured to present medication schedules, reminders, adherence reports, health-score information, dispensing confirmations, snooze options, configuration screens, and placement-confirmation interfaces.

The peripheral data source 106 may include a wearable device, medical monitoring device, fitness device, smart sensor, or other connected data source associated with the user 102. The peripheral data source 106 may provide biometric signals, activity signals, sleep signals, device telemetry, contextual information, or other health-related signals used by the system 100 to support health-context computation and personalized medication adherence control.

The backend system 108 may include one or more processors 110, one or more non-transitory storage media 112, a communications interface 114, a wireless interface 116, a trained inference model 118, a pharmacological reference data store 120, an empty-container weight data store 122, a jurisdictional compliance rule set 124, an event record store 126, and a composite identifier 128. The backend system 108 may be implemented as a cloud system, local system, on-device system, distributed system, or any combination thereof. The backend system 108 may perform prescription extraction, prescription validation, user-profile management, device-profile management, medication-regimen management, control-instruction generation, wellness-score computation, weight-based verification, reminder escalation, event logging, notification routing, and synchronization with the medication dispensing apparatus 130 and the user client device 104.

The one or more processors 110 may execute instructions stored in the one or more non-transitory storage media 112 to perform operations of the backend system 108. The one or more non-transitory storage media 112 may store user profile data, device identifiers, prescription records, medication placement records, medication schedules, event records, configuration data, calibration data, health-score data, contextual factor data, medication interaction data, contraindication data, and system logs. The communications interface 114 may transmit a control instruction 150 to the medication dispensing apparatus 130, receive an event record 152 from the medication dispensing apparatus 130, exchange configuration or synchronization data, and transmit a notification 154 to one or more recipient devices 132. The wireless interface 116 may receive or relay data from the peripheral data source 106 and may support pairing, discovery, authentication, or telemetry exchange.

The trained inference model 118 may process image data captured by the image sensor 140 to generate a structured prescription record 146. The trained inference model 118 may include an AI/ML engine, optical-character-recognition component, image-processing component, language-processing component, field-extraction component, rule-based parser, statistical model, machine-learning model, or hybrid inference pipeline. The image sensor 140 may be part of the user client device 104, the medication dispensing apparatus 130, a dedicated scanning device, or another image-capture device. The image sensor 140 may capture a single image, multiple images, a burst sequence, a video stream, or a multi-angle image sequence of a prescription label associated with a medication container.

The pharmacological reference data store 120 may validate extracted prescription fields, including medication name, dosage, frequency, timing, duration, interaction condition, contraindication condition, or other medication-related information. The jurisdictional compliance rule set 124 may support localization or formatting of the structured prescription record 146, including dosage units, language, date format, regional medication naming, or presentation requirements. The empty-container weight data store 122 may store reference weights for medication containers, vial types, bottle categories, packaging types, or other container categories, and may support estimation of an initial-units quantity when such quantity is unavailable from the structured prescription record 146.

The event record store 126 may store event records 152 generated in connection with medication loading, device pairing, prescription scanning, placement confirmation, scheduled dispensing, access authorization, compartment opening, medication retrieval, weight measurement, reminder activity, snooze response, escalation, refill forecasting, or other system events. Event records may be stored locally, remotely, in the cloud, on the medication dispensing apparatus 130, on the user client device 104, or across distributed storage resources. In some implementations, event records may be encrypted, access-controlled, synchronized, backed up, or integrity-protected. In some implementations, event records may be encrypted, access-controlled, synchronized, backed up, or otherwise protected. Alternatively, cryptographic binding may be used as an optional implementation, but it is not required in every embodiment.

The composite identifier 128 may identify or link a medication-specific operating context of the user 102. In some implementations, the composite identifier 128 may implement, extend, or operate together with a Health Record Identifier or unique digital record associated with the user 102. The composite identifier 128 may be generated based at least in part on a user profile record 142, a device identifier 144, the structured prescription record 146, and a placement record 148. In other implementations, the composite identifier 128 may additionally or alternatively reference a user identifier, device identifier, medication identifier, regimen identifier, node identifier, compartment identifier, storage-device identifier, event identifier, or other record that links user context, device context, prescription context, and medication-storage context.

The user profile record 142 may include personal identifiers, demographic information, biometric baseline information, medical history, allergy information, dietary information, caregiver information, emergency-contact information, regular vital signs, lifestyle patterns, fitness routines, geographic or environmental information, medication-regimen preferences, or other health-profile information. The device identifier 144 may identify the peripheral data source 106, the user client device 104, the medication dispensing apparatus 130, a controller unit, a node unit, or another device associated with the system 100. The device identifier 144 may be obtained through QR code scanning, manual entry, Bluetooth discovery, Wi-Fi discovery, NFC interaction, RFID reading, serial number entry, or another device-onboarding technique.

The structured prescription record 146 may include prescription information extracted, entered, validated, or localized during medication-regimen acquisition. The structured prescription record 146 may include drug name, prescription identifier, dosage, regimen, frequency, timing, duration, prescribing physician information, refill information, special instructions, medication identifier, initial-units field, or additional metadata. The placement record 148 may identify physical placement of a medication container in the medication dispensing apparatus 130, including placement in a compartment, drawer, node unit, carousel bay, vial receptacle, slot, tray, or other medication-container receiving region. The placement record 148 may be generated by user confirmation, device-display selection, image recognition, RFID, NFC, QR scanning, node identifier, compartment identifier, caregiver confirmation, or controller feedback.

The medication dispensing apparatus 130 may be implemented as a carousel-based apparatus, drawer-based apparatus, node-based apparatus, cabinet-based apparatus, tray-based apparatus, modular medication storage device, smart medication storage device, or another controlled-access medication storage device. The medication dispensing apparatus 130 may include one or more compartments, and each compartment may be implemented as a carousel compartment, drawer, bay, bin, slot, vial receptacle, node unit, storage drawer, or other medication-container receiving region. The medication dispensing apparatus 130 may include locking mechanisms, actuators, smartlocks, motors, reed switches, position sensors, weight sensors, LEDs, display units, speakers, microphones, communication modules, and local storage. Thus, the medication dispensing apparatus 130 may cover both carousel-based implementations and drawer/node-based implementations disclosed for smart medication storage devices.

The recipient devices 132 may include a caregiver device 134, a healthcare provider device 136, an emergency contact device 138, or another device associated with an authorized recipient. The backend system 108 may transmit the notification 154 to one or more recipient devices 132 in response to a missed dose, delayed confirmation, medication overuse, medication underuse, low medication level, failed confirmation, tampering indication, escalation state, or other event.

In operation, the system 100 may initialize the user profile record 142, onboard the peripheral data source 106 or medication dispensing apparatus 130 using device identifiers, capture or receive prescription information, generate the structured prescription record 146, and generate the placement record 148 when the medication container is assigned to a physical storage region. The backend system 108 may generate or associate the composite identifier 128 with these records. When a dispensing event is due, the backend system 108 may generate the control instruction 150 addressing a designated compartment of the medication dispensing apparatus 130, wherein the control instruction 150 may be conditioned on correspondence between the placement record 148 and the designated compartment. The medication dispensing apparatus 130 may then unlock, open, rotate, expose, illuminate, extend, or otherwise present the designated compartment. After the physical state change, the medication dispensing apparatus 130 may provide the event record 152 indicating the outcome of the physical state change, sensor readings, confirmation status, reminder status, access status, or adherence status.

In some implementations, the system 100 may compute a Personalized Health Score, wellness score, or other health-context value using biometric signals from the peripheral data source 106, user context from the user profile record 142, medication-regimen information from the structured prescription record 146, and prior event records 152. The score may be updated over time based on real-time health data, contextual inputs, medication adherence events, sensor feedback, and user behavior. The resulting score may be presented to the user 102, used to generate reports, used to route notifications, or used to influence a subsequent control instruction.

### Composite Identifier and Operational Pipeline

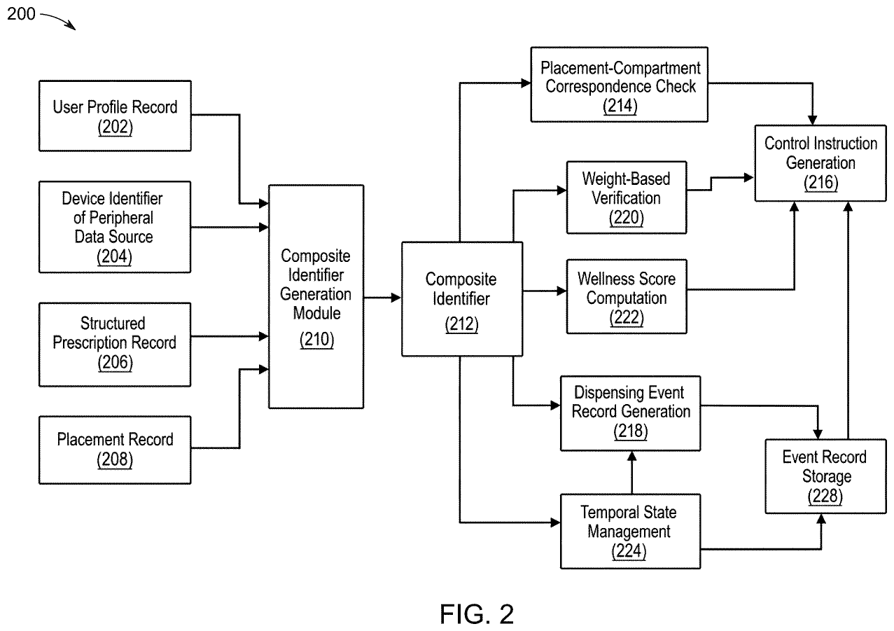

FIG. 2 illustrates a composite identifier generation and operational module pipeline 200 of the system 100 of FIG. 1, in accordance with an embodiment. The pipeline 200 provides a coordinated data and control framework in which user information, device information, prescription information, medication placement information, dispensing-control logic, sensor feedback, health-score computation, reminder escalation, and event-record storage operate together. The pipeline 200 may be performed by the backend system 108, the medication dispensing apparatus 130, the user client device 104, a distributed computing system, or a combination thereof.

The pipeline 200 includes a user profile record 202, a device identifier of a peripheral data source 204, a structured prescription record 206, and a placement record 208. These records may reflect four primary factors used to create a medication-specific operating context: the user, the paired device or data source, the medication prescription, and the physical placement of the medication container. This identity-mapping approach may implement, extend, or operate together with a Health Record Identifier or unique digital record associated with the user. In the illustrated embodiment, the records are provided to a composite identifier generation module 210 to generate a composite identifier 212.

The user profile record 202 may include personal identifiers, vital signs, biometric baseline values, medical history, dietary information, fitness information, lifestyle patterns, caregiver information, emergency contacts, medication preferences, geographic location, environmental exposure, and other health-profile data. The user profile record 202 may be generated or updated through a user-facing application, caregiver interface, provider interface, backend database, or device-driven data collection process.

The device identifier of a peripheral data source 204 may identify a wearable, sensor, medical monitoring device, mobile device, controller unit, node unit, medication dispensing apparatus, or other peripheral data source associated with the user. The device identifier 204 may be obtained through QR code scanning, manual entry, Bluetooth discovery, Wi-Fi discovery, NFC interaction, RFID reading, factory-embedded hardware identifier, or another secure onboarding method. In some implementations, the system creates a one-to-one mapping between the user and each device so that biometric signals, medication-device events, and dispensing events are associated with the correct user and device context.

The structured prescription record 206 may represent the prescription-related portion of the pipeline 200. The structured prescription record 206 may be generated from manual entry, prescription scanning, uploaded prescription images, captured prescription-label images, pharmacy records, provider updates, or another prescription-acquisition technique. In some implementations, the structured prescription record 206 may be obtained by applying a trained inference model to image data of a prescription label, including multiple images captured while rotating a medication container relative to an image sensor. The structured prescription record 206 may include drug name, dosage, frequency, timing, duration, prescription identifier, prescribing physician details, regimen information, special instructions, refill data, medication identifier, initial-units field, and additional metadata. The structured prescription record 206 may be validated against medicinal, pharmacological, clinical, or compliance reference data.

The placement record 208 may represent the physical medication-placement portion of the pipeline 200. The placement record 208 may indicate that a medication container associated with the structured prescription record 206 has been assigned to a designated compartment of the medication dispensing apparatus 130. The designated compartment may be a carousel compartment, drawer, node unit, bay, bin, slot, vial receptacle, tray, or other medication-container receiving region. The placement record 208 may be generated when a user selects a touchpoint on a display and places the medication container into a corresponding physical location. The placement record 208 may also be generated or confirmed using RFID, NFC, QR code scanning, image recognition, compartment selection, node identifier, controller identifier, user confirmation, or caregiver confirmation.

The composite identifier generation module 210 generates the composite identifier 212 based at least in part on the user profile record 202, the device identifier of the peripheral data source 204, the structured prescription record 206, and the placement record 208. In some implementations, the composite identifier 212 may be generated from all four records. In other implementations, the composite identifier 212 may be generated from fewer records, additional records, or alternative records, provided that the generated identifier preserves an association among the user context, device context, medication context, and medication storage context. The composite identifier 212 may be stored as a structured tuple, database key, linked data record, secure identifier, or another data structure suitable for associating downstream operations with a medication-specific context.

The composite identifier 212 may function as a medication-dispensing-specific extension of a Health Record Identifier. For example, the Health Record Identifier may identify a user's health context using user data, biometric signals, and health history, while the composite identifier 212 may further associate that health context with a particular medication, prescription, storage device, and physical placement. In this manner, the composite identifier 212 may support both personalized health computation and traceable medication dispensing.

The placement-compartment correspondence check 214 determines whether the placement record 208 corresponds to the designated compartment addressed by a proposed control instruction. This check may compare a compartment identifier, node identifier, drawer identifier, carousel position, medication container identifier, medication identifier, user identifier, prescription identifier, or device identifier. In a carousel-based implementation, the placement-compartment correspondence check 214 may determine whether the medication container identified by the placement record 208 is assigned to the carousel compartment that will be rotated to the dispensing position. In a node-based or drawer-based implementation, the check may determine whether the medication container identified by the placement record 208 is stored in the node, drawer, or smartlocked compartment that will be unlocked or opened.

The control instruction generation 216 generates a control instruction addressing a designated compartment. The control instruction may include an instruction code, compartment identifier, node identifier, drawer identifier, carousel position, actuator command, lock command, door command, LED command, timestamp, medication identifier, user identifier, composite identifier 212, or another control parameter. In some implementations, the control instruction generation 216 occurs only after the placement-compartment correspondence check 214 confirms that the placement record 208 corresponds to the designated compartment addressed by the control instruction. Thus, the control instruction performs a physical control function that causes a selected compartment, drawer, node, or carousel bay to become accessible only when the corresponding placement condition is satisfied.

The dispensing event record generation 218 generates an event record associated with a dispensing operation or attempted dispensing operation. The event record may include the composite identifier 212, a timestamp, medication identifier, user identifier, compartment identifier, drawer identifier, node identifier, physical state-change outcome, lock state, open/closed state, sensor status, user confirmation status, reminder state, snooze response, weight measurement, adherence status, error status, or notification status. The event record may be stored locally, stored in the backend system, synchronized with cloud storage, associated with the user's adherence history, used to update a wellness score, or transmitted to a caregiver or healthcare provider.

The weight-based verification 220 processes weight data or sensor data associated with the medication dispensing apparatus. In some implementations, the weight-based verification 220 receives a baseline container weight measurement and a current container weight measurement from a weight sensor or load cell. The weight-based verification 220 may compute a weight differential, determine or retrieve a per-unit mass, estimate consumed medication units, determine an initial-units quantity, estimate remaining units, forecast refill timing, detect missed medication, detect overuse, detect possible misuse, detect tampering, or flag an anomaly. The output of the weight-based verification 220 may influence later dispensing control, notification routing, adherence reports, refill alerts, or wellness-score updates.

The wellness score computation 222 computes a wellness score, Personalized Health Score, health-context value, or other normalized health indicator associated with the user. The wellness score computation 222 may receive biometric signals from the peripheral data source associated with the device identifier 204 and may combine those signals with contextual information from the user profile record 202, regimen information from the structured prescription record 206, and event data from prior dispensing records. The wellness score computation 222 may normalize biometric signals and weight the normalized values using contextual factors, such as medication regimen, activity level, symptom severity, time of day, deviation from historical baseline, geographic or environmental factor, and health profile.

The temporal state management 224 manages scheduled dispensing times, reminder states, snooze responses, output patterns, escalation thresholds, and recipient notifications. For a scheduled dispensing event, the temporal state management 224 may define a sequence of temporal states ordered by increasing elapsed time from a scheduled medication time. Each temporal state may be associated with a distinct output pattern, including a display prompt, LED indication, audio reminder, haptic cue, push notification, mobile message, caregiver message, healthcare provider message, or other user-facing or recipient-facing output. A snooze response may reset the sequence to an earlier state a bounded number of times. If the user does not confirm medication intake within a defined time window, the temporal state management 224 may transmit a notification to a caregiver, healthcare provider, emergency contact, or other recipient.

The event record storage 228 stores the event records generated by the dispensing event record generation 218, weight-based verification 220, wellness score computation 222, or temporal state management 224. The event record storage 228 may include embedded storage, controller storage, user client storage, backend storage, cloud storage, distributed storage, or combinations thereof. The event record storage 228 may store records of medication cycles, user interactions, access authorization, compartment opening, node unlocking, sensor readings, weight changes, confirmation status, snooze events, reminder escalation, notifications, refill forecasts, or wellness-score updates. The stored event records may support adherence monitoring, caregiver review, healthcare-provider reporting, user feedback, medication management, and later system analytics.

In an example operation of the pipeline 200, the system receives or retrieves the user profile record 202, obtains the device identifier 204 for a paired peripheral data source, generates the structured prescription record 206 from scanned or entered prescription information, and obtains the placement record 208 when the medication container is physically placed in a compartment. The composite identifier generation module 210 generates the composite identifier 212 from these inputs. When a scheduled dispensing event occurs, the placement-compartment correspondence check 214 verifies that the placement record 208 corresponds to the compartment to be addressed. If the correspondence exists, the control instruction generation 216 generates a control instruction. The medication dispensing apparatus then performs a physical state change. The dispensing event record generation 218 generates an event record. The weight-based verification 220 may verify medication consumption. The wellness score computation 222 may update the wellness score. The temporal state management 224 may manage reminders and escalation. The event record storage 228 stores the event record linked to the composite identifier 212.

The pipeline 200 thereby provides a more explicit control architecture for physical medication dispensing. The user information module corresponds to the user profile record 202 and health-score inputs. The device information module corresponds to the device identifier 204 and peripheral data source integration. The prescription information module corresponds to the structured prescription record 206. The final medication placement and node assignment workflow corresponds to the placement record 208. The Health Record Identifier or unique digital record concept corresponds to, or may be extended by, the composite identifier 212. The node/drawer-based smart medication storage device and the carousel-based medication dispensing apparatus may both operate under this pipeline because both implementations use placement, control, sensor feedback, and event-record association.

### Carousel-Based Medication Dispensing Apparatus

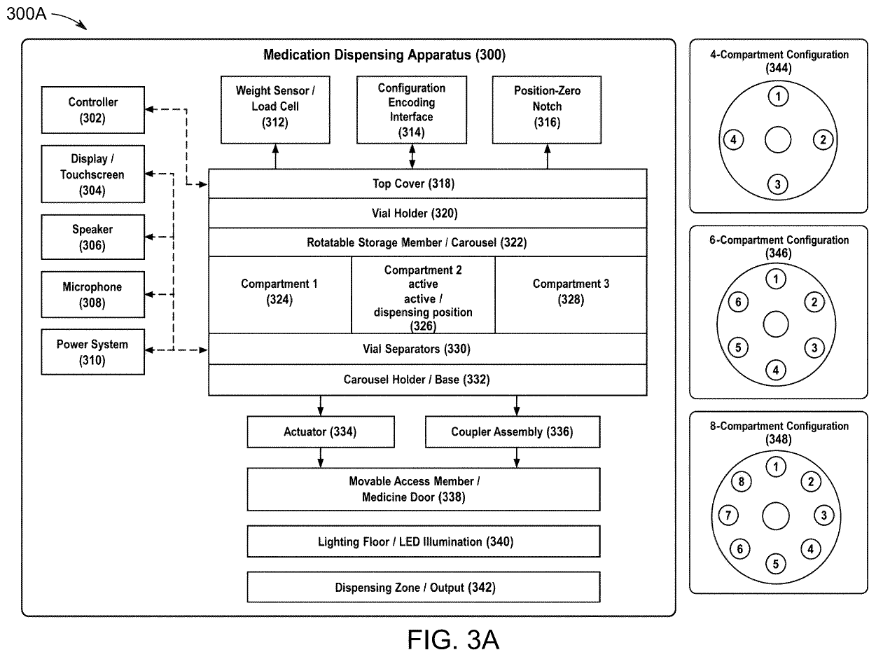

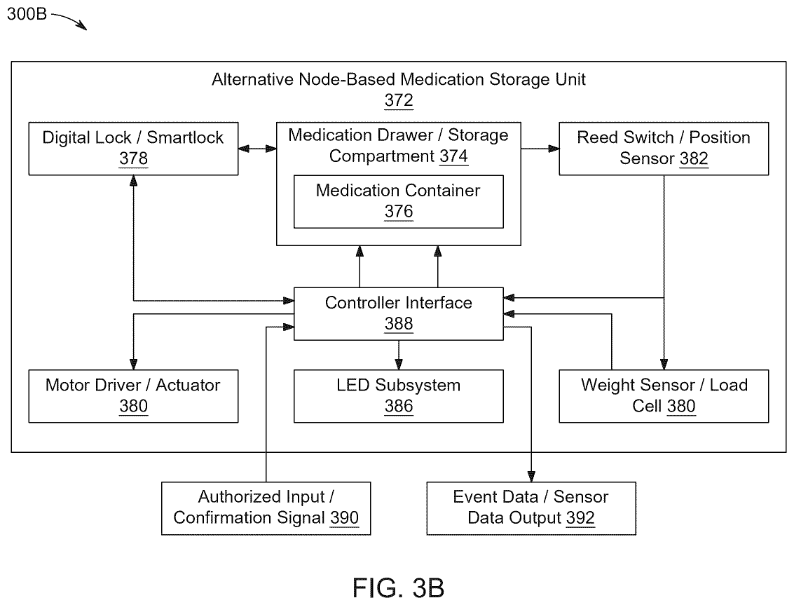

FIG. 3A illustrates a medication dispensing apparatus 300 having a carousel-based dispensing architecture, in accordance with an embodiment. The medication dispensing apparatus 300 may implement the medication dispensing apparatus 130 of FIG. 1 and may operate under the composite-identifier-based control pipeline described with reference to FIG. 2. The medication dispensing apparatus 300 is one example of a controlled-access medication storage device having a plurality of medication-container receiving regions. The medication-container receiving regions may be implemented as carousel compartments, drawers, bays, bins, slots, vial receptacles, node-based units, or other secure storage regions configured to receive one or more medication containers.

The medication dispensing apparatus 300 includes a controller 302 configured to coordinate sensing, actuation, user-interface output, user confirmation, and event-record generation. The controller 302 may receive a control instruction from the backend system 108, determine a designated compartment addressed by the control instruction, and cause a physical state change of the designated compartment. The controller 302 may also receive sensor data, generate local event data, synchronize event data with the backend system 108, and coordinate operation of a display/touchscreen 304, a speaker 306, a microphone 308, a power system 310, a weight sensor/load cell 312, a configuration-encoding interface 314, and a position-zero notch 316.

The display/touchscreen 304 may present medication schedules, dispensing prompts, medication reminders, adherence messages, error messages, confirmation screens, snooze options, emergency access prompts, configuration menus, and device-status information. The display/touchscreen 304 may also receive user inputs, including a dispensing confirmation, placement confirmation, snooze response, emergency request, or access authorization. The speaker 306 may generate audible reminders, alert sounds, spoken medication instructions, caregiver messages, or confirmation tones. The microphone 308 may receive voice inputs, voice confirmations, emergency requests, caregiver messages, or other audio inputs.

The power system 310 supplies power to the medication dispensing apparatus 300 and may include one or more batteries, charging circuits, wired power inputs, voltage regulators, power-management circuits, and backup-power components. In some implementations, the power system 310 allows the medication dispensing apparatus 300 to continue selected operations during a power interruption, including storage-state retention, logging, reminder output, emergency access, or synchronization after reconnection.

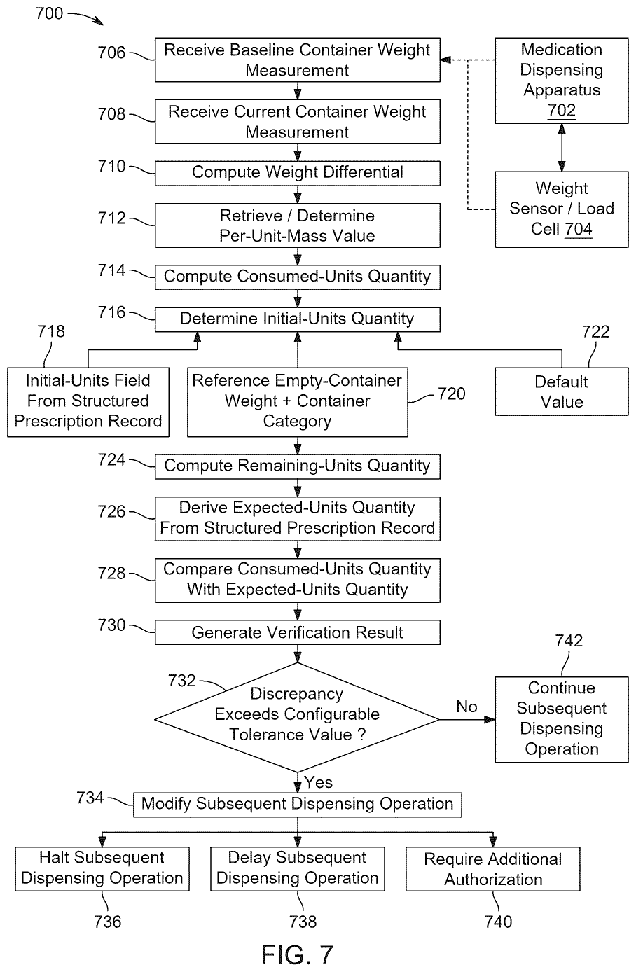

The weight sensor/load cell 312 may measure a weight associated with the medication dispensing apparatus 300, a medication container, a support surface, or a compartment. The weight sensor/load cell 312 may generate baseline and current weight measurements before and after a dispensing event. The controller 302 or backend system 108 may use the measurements to compute a weight differential, estimate consumed units, estimate remaining units, detect medication removal, detect a missed dose, detect over-consumption, detect potential tampering, or forecast refill timing. Thus, the weight sensor/load cell 312 provides physical sensor support for the weight-based verification described with reference to FIG. 7.

The configuration-encoding interface 314 identifies an installed compartment configuration of the medication dispensing apparatus 300. For example, the configuration-encoding interface 314 may communicate whether the apparatus is configured as a four-compartment configuration 344, a six-compartment configuration 346, an eight-compartment configuration 348, or another compartment configuration. The controller 302 may use the installed compartment count to set indexing of the plurality of compartments and to correlate each logical compartment identifier with a corresponding physical position.

The position-zero notch 316 provides a home-reference feature for indexing the rotatable storage member/carousel 322. The position-zero notch 316 may cooperate with an optical sensor, magnetic sensor, Hall-effect sensor, mechanical switch, proximity sensor, or other position-detection element. The controller 302 may use the position-zero notch 316 during power-on, reset, calibration, reassembly, or emergency-access recovery to determine a known angular reference position of the rotatable storage member/carousel 322.

The medication dispensing apparatus 300 further includes a top cover 318, a vial holder 320, the rotatable storage member/carousel 322, a plurality of compartments 324, 326, 328, vial separators 330, and a carousel holder/base 332. The top cover 318 may enclose, protect, align, or provide access to the medication storage region. The vial holder 320 may support or retain medication containers relative to the rotatable storage member/carousel 322. The rotatable storage member/carousel 322 carries the plurality of compartments and rotates to place a selected compartment at a dispensing position.

In the illustrated embodiment, the plurality of compartments includes a first compartment 324, a second compartment 326, and a third compartment 328. The second compartment 326 is shown as an active compartment at a dispensing position and may correspond to a designated compartment addressed by a control instruction. The first compartment 324 and the third compartment 328 represent additional medication-container receiving regions. Although three compartments are shown for clarity, the medication dispensing apparatus 300 may include any suitable number of compartments.

The vial separators 330 define boundaries between adjacent compartments. The vial separators 330 may be fixed, removable, or reconfigurable and may maintain separation, alignment, and stability of medication containers during rotation. The carousel holder/base 332 supports the rotatable storage member/carousel 322 and may provide mechanical stability, rotational alignment, and a physical reference between the storage member and the dispensing zone/output 342.

The actuator 334 drives movement of the rotatable storage member/carousel 322 or another movable portion of the medication dispensing apparatus 300. The actuator 334 may include a servo motor, stepper motor, geared motor, direct-drive motor, solenoid, or other electromechanical actuator. The coupler assembly 336 mechanically couples the actuator 334 to the rotatable storage member/carousel 322 or another driven component and may transfer torque, maintain angular alignment, assist reassembly, or preserve a known drive relationship.

The movable access member/medicine door 338 selectively exposes the active compartment at the dispensing position while restricting access to non-designated compartments. In response to a control instruction, the controller 302 may cause the actuator 334, the coupler assembly 336, or a separate door actuator to transition the movable access member/medicine door 338 from a closed state to an open or accessible state. In this way, the control instruction causes or enables a physical state change of the designated compartment.

The lighting floor/LED illumination 340 may illuminate the active compartment, the dispensing zone/output 342, or a user-facing portion of the medication dispensing apparatus 300. The lighting floor/LED illumination 340 may also provide status feedback, reminder feedback, missed-dose feedback, low-medication feedback, configuration feedback, error feedback, or escalation feedback. The drawing may show the lighting floor/LED illumination 340 in black-and-white form, while the specification may describe visual patterns, intensities, or colors as optional examples.

The dispensing zone/output 342 is positioned to present the active compartment, medication container, or medication access region to the user. The dispensing zone/output 342 may include an opening, tray, aperture, retrieval area, door region, or other accessible region. The controller 302 may record event data when the active compartment reaches the dispensing zone/output 342, when the movable access member/medicine door 338 opens, when the user confirms the medication event, or when the weight sensor/load cell 312 detects a weight change.

The four-compartment configuration 344, six-compartment configuration 346, and eight-compartment configuration 348 illustrate example installed configurations. These configurations support the later-developed carousel product design while preserving the broader concept of a medication dispensing apparatus having multiple medication-container receiving regions. The configuration-encoding interface 314 may communicate the installed configuration to the controller 302 so that the controller 302 indexes the compartments properly and maintains correspondence between a placement record and the physical compartment addressed by a control instruction.

During an example operation, the controller 302 receives a control instruction identifying the second compartment 326 as the designated compartment. The controller 302 uses the configuration-encoding interface 314 and the position-zero notch 316 to determine the correct rotational position. The actuator 334 rotates the rotatable storage member/carousel 322 to position the second compartment 326 at the dispensing zone/output 342. The movable access member/medicine door 338 then exposes the second compartment 326. The weight sensor/load cell 312, display/touchscreen 304, speaker 306, microphone 308, lighting floor/LED illumination 340, and other sensors or outputs may participate in the resulting dispensing event. The controller 302 may generate event data and provide such event data to the backend system 108 for storage in association with the composite identifier.

Although FIG. 3A illustrates a carousel-based medication dispensing apparatus 300, the disclosed medication dispensing apparatus is not limited to a carousel configuration. In other implementations, the plurality of compartments may be implemented as drawer-based compartments, node-based compartments, smartlock compartments, bins, bays, slots, vial receptacles, or other storage regions. FIG. 3B illustrates one such node-based embodiment. Accordingly, the carousel-based architecture of FIG. 3A and the node-based architecture of FIG. 3B may both operate under the same prescription-extraction, placement-record, composite-identifier, weight-verification, and event-recording framework.

### Node-Based Medication Storage Unit

FIG. 3B illustrates an alternative node-based medication storage unit 372, in accordance with an embodiment. The node-based medication storage unit 372 may implement one compartment, one storage region, or a plurality of compartments of the medication dispensing apparatus 130 of FIG. 1. In some implementations, multiple node-based medication storage units 372 may be arranged together to form a multi-compartment medication dispensing apparatus. In other implementations, the node-based medication storage unit 372 may operate as a drawer, bay, bin, slot, chamber, or secure compartment within a larger medication dispensing apparatus.

The node-based embodiment may include a secure enclosure, a digitally actuated lock, a medication drawer or storage compartment, LED status output, a reed switch or position sensor, a motorized drawer mechanism, a weight sensor/load cell, and a controller interface. The node-based embodiment may remain locked until a scheduled medication time and may open only after an authorized confirmation is received.

The node-based medication storage unit 372 includes a medication drawer/storage compartment 374 configured to receive a medication container 376. The medication drawer/storage compartment 374 may be implemented as a drawer, bay, bin, slot, chamber, tray, receptacle, or enclosed storage region. The medication container 376 may include a bottle, vial, blister pack, cartridge, pouch, or other medication container. The medication drawer/storage compartment 374 may hold the medication container 376 in a position suitable for access, sensing, weighing, or controlled presentation to the user.

The digital lock/smartlock 378 controls access to the medication drawer/storage compartment 374. The digital lock/smartlock 378 may include an electronically actuated latch, solenoid lock, motorized lock, magnetic lock, electromechanical latch, or other controllable lock mechanism. The digital lock/smartlock 378 may maintain the medication drawer/storage compartment 374 in a locked state outside an authorized dispensing window. In some implementations, the digital lock/smartlock 378 transitions to an unlocked state only when a scheduled medication time has occurred and the controller interface 388 has received an authorized input/confirmation signal 390.

The motor driver/actuator 380 may drive opening, closing, extension, retraction, release, or positioning of the medication drawer/storage compartment 374. The motor driver/actuator 380 may operate with or independently from the digital lock/smartlock 378. For example, the digital lock/smartlock 378 may release the medication drawer/storage compartment 374, and the motor driver/actuator 380 may move the medication drawer/storage compartment 374 to an accessible position. In another example, the motor driver/actuator 380 may operate a sliding, hinged, translational, or lifting access mechanism.

The reed switch/position sensor 382 detects a position or access state of the medication drawer/storage compartment 374. The reed switch/position sensor 382 may detect whether the medication drawer/storage compartment 374 is open, closed, partially extended, fully extended, locked, unlocked, or otherwise positioned relative to a housing. The reed switch/position sensor 382 may include a magnetic reed switch, Hall-effect sensor, optical sensor, limit switch, proximity sensor, encoder, or another position-detection element. Drawer movement or drawer extension may also be determined using motor runtime, pulse count, switch state, sensor feedback, or a combination thereof.

The weight sensor/load cell 384 measures a weight associated with the medication container 376, the medication drawer/storage compartment 374, or a support surface on which the medication container 376 rests. The weight sensor/load cell 384 may generate before-and-after weight data associated with a dispensing event. For example, the weight sensor/load cell 384 may measure a baseline weight before the user accesses the medication container 376 and a current weight after the dispensing event. The controller interface 388 or backend system 108 may use the before-and-after weight data to compute a weight differential, estimate consumed units, estimate remaining units, detect a missed medication event, detect an over-consumption event, detect possible tampering, or support refill forecasting.

The LED subsystem 386 provides visual status feedback associated with the node-based medication storage unit 372. The LED subsystem 386 may indicate ready-to-dispense, locked, unlocked, open, closed, missed dose, low medication level, error, synchronization status, escalation status, or another operational state. The LED subsystem 386 may operate in coordination with reminder timing, authorized confirmation, drawer movement, sensor feedback, and event-record generation.

The controller interface 388 coordinates operation of the node-based medication storage unit 372. The controller interface 388 may receive the authorized input/confirmation signal 390, receive status signals from the reed switch/position sensor 382, receive weight data from the weight sensor/load cell 384, actuate the digital lock/smartlock 378, drive the motor driver/actuator 380, and control the LED subsystem 386. The controller interface 388 may be implemented as a local node controller, controller bus, processor interface, wireless interface, wired interface, or interface to the controller 302 of FIG. 3A.

The authorized input/confirmation signal 390 represents authorization or confirmation used to permit access to the medication drawer/storage compartment 374. The authorized input/confirmation signal 390 may be generated from biometric confirmation, mobile-device confirmation, touchscreen input, caregiver confirmation, passcode input, proximity-based authentication, voice confirmation, or another authorized input. In some implementations, the authorized input/confirmation signal 390 is evaluated together with a scheduled medication time before the controller interface 388 actuates the digital lock/smartlock 378 or the motor driver/actuator 380.

The event data/sensor data output 392 represents data generated by the node-based medication storage unit 372 and provided to another system component. The event data/sensor data output 392 may be provided to the backend system 108, the controller 302, the event record store 126, or another local or remote storage component. The event data/sensor data output 392 may include a drawer-open event, drawer-closed event, lock-state event, authorization event, motor-actuation event, reed-switch state, position-sensor state, baseline weight measurement, current weight measurement, weight differential, estimated consumed-units quantity, estimated remaining-units quantity, LED status, error status, missed-dose status, tamper status, or adherence status.

In an example operation, the node-based medication storage unit 372 remains locked by the digital lock/smartlock 378 until a scheduled medication time occurs. After the authorized input/confirmation signal 390 is received, the controller interface 388 actuates the digital lock/smartlock 378 and drives the motor driver/actuator 380 to permit access to the medication drawer/storage compartment 374. The reed switch/position sensor 382 detects opening, closing, or extension of the medication drawer/storage compartment 374. The weight sensor/load cell 384 measures the medication container 376 before and after the dispensing event. The LED subsystem 386 provides visual status output. The controller interface 388 generates the event data/sensor data output 392 for storage, verification, synchronization, or later analysis.

In the carousel embodiment of FIG. 3A, a control instruction may cause a physical state change by rotating a carousel and exposing an active compartment. In the node-based embodiment of FIG. 3B, a control instruction may cause a physical state change by unlocking, opening, extending, or otherwise providing access to the medication drawer/storage compartment 374. In both embodiments, the physical state change may be linked to a placement record, sensor feedback, an event record, and the composite identifier.

### Electrical and Computational Architecture

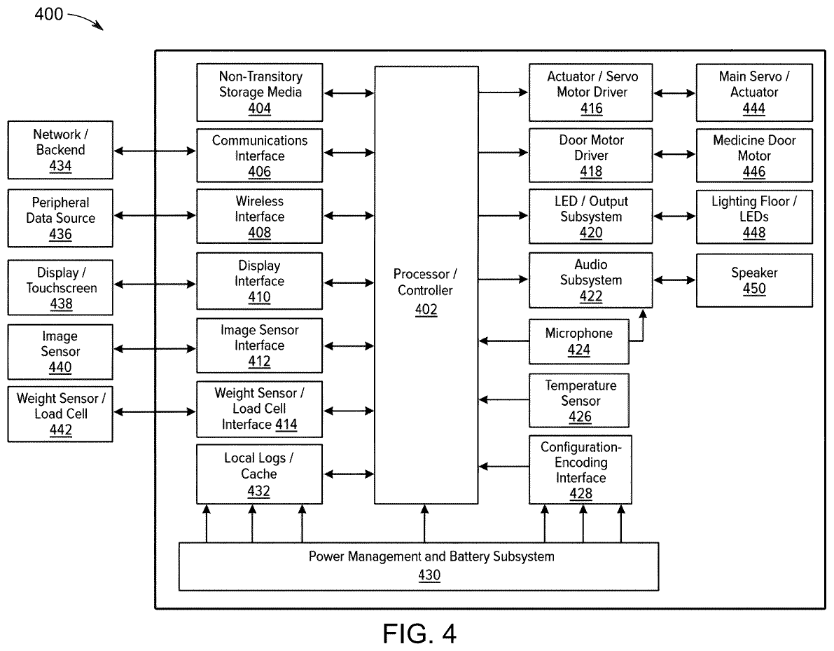

FIG. 4 illustrates an electrical and computational architecture 400 of a medication dispensing apparatus, in accordance with an embodiment. The architecture 400 may implement the electronics and control architecture of the carousel-based medication dispensing apparatus 300 of FIG. 3A, the node-based medication storage unit 372 of FIG. 3B, a drawer-based apparatus, a smart medication storage device, or another controlled-access medication storage apparatus. The architecture 400 may also implement firmware and hardware interactions disclosed for the controller unit, node unit, display unit, sensors, communication modules, and storage components.

The architecture 400 includes a processor/controller 402. The processor/controller 402 may execute firmware or software instructions stored on non-transitory storage media 404. The processor/controller 402 may receive control instructions, generate control outputs, drive actuators, control access mechanisms, receive sensor inputs, manage reminders, process local event data, control user-interface outputs, manage local logs/cache 432, and communicate event data to a network/backend 434. The processor/controller 402 may include a microcontroller, processor, system-on-chip, embedded controller, or other control circuit.

The non-transitory storage media 404 may store firmware, configuration data, calibration data, device identifiers, local logs, event data, sensor data, reminder settings, schedule information, and temporary session data. In some implementations, the non-transitory storage media 404 may store local cached records when a network connection is unavailable and may later synchronize such records with the network/backend 434.

The communications interface 406 couples the processor/controller 402 to the network/backend 434. The communications interface 406 may exchange control instructions, event records, medication schedules, configuration information, firmware update data, logs, reminders, and adherence information with the backend system 108. The communications interface 406 may be wired or wireless and may support local, wide-area, or Internet-based communication.

The wireless interface 408 may communicate with a peripheral data source 436, a user client device, a wearable device, a medical sensor, a node unit, a controller unit, or another wireless device. The wireless interface 408 may support pairing, device discovery, device authentication, biometric data transfer, or short-range communication. In some implementations, biometric signals received through the wireless interface 408 may be forwarded to the backend system 108 for wellness-score or Personalized Health Score computation.

The display interface 410 drives a display/touchscreen 438. The display/touchscreen 438 may provide medication reminders, adherence feedback, next-medication preview, setup screens, error alerts, system status, confirmation prompts, snooze prompts, emergency access prompts, firmware update notifications, offline-mode notifications, or other user-interface screens. The display interface 410 may also receive touch input and provide corresponding input data to the processor/controller 402.

The image sensor interface 412 receives image data from an image sensor 440. The image sensor 440 may capture prescription-label images, QR codes, medication-container images, device-pairing codes, compartment-state images, or other visual input. The image sensor interface 412 may provide the image data to the processor/controller 402 or to the network/backend 434 for prescription extraction, device onboarding, placement verification, or event confirmation.

The weight sensor/load cell interface 414 receives weight measurements from a weight sensor/load cell 442. The weight sensor/load cell interface 414 may receive baseline and current weight measurements before and after a dispensing event. The weight measurements may support consumed-units estimation, remaining-units estimation, missed-dose detection, over-consumption detection, low-medication detection, refill forecasting, and anomaly detection.

The actuator/servo motor driver 416 drives a main servo/actuator 444. The main servo/actuator 444 may rotate a carousel, position a compartment, move a storage member, index a vial holder, or drive another movable structure. The door motor driver 418 drives a medicine door motor 446. The medicine door motor 446 may open or close a movable access member, medicine door, drawer, shutter, smartlock, or other access mechanism. In some implementations, the actuator/servo motor driver 416 and the door motor driver 418 cooperate to expose only a designated compartment while restricting access to other compartments.

The LED/output subsystem 420 may drive lighting floor/LEDs 448 or another visual output element. The LED/output subsystem 420 may provide reminder indication, active-compartment indication, low-medication indication, missed-dose indication, error indication, synchronization indication, or temporal-state output patterns. The audio subsystem 422 may drive a speaker 450 to provide reminder tones, audio instructions, caregiver messages, alerts, or confirmation sounds. The microphone 424 may receive user voice input, emergency requests, spoken confirmations, caregiver messages, or other audio input.

The temperature sensor 426 may provide environmental or internal temperature information. The temperature information may support device monitoring, sensor compensation, medication-storage monitoring, environmental context, or safety alerts. The configuration-encoding interface 428 may identify an installed compartment configuration, carousel configuration, node configuration, drawer count, vial configuration, or another physical configuration of the medication dispensing apparatus. The processor/controller 402 may use configuration information to set compartment indexing and control addressing.

The power-management and battery subsystem 430 supplies regulated power to the architecture 400 and may include battery-management circuitry, charging circuitry, voltage regulation, backup power, charge-status monitoring, and power-failure detection. The local logs/cache 432 may store recent event records, sensor readings, operational logs, medication schedules, reminder history, synchronization status, or temporary data. The local logs/cache 432 may synchronize with the network/backend 434 when communication is available.

In operation, the processor/controller 402 receives a control instruction from the network/backend 434, identifies the designated compartment or storage region, and controls the appropriate subsystem to cause a physical state change. In a carousel embodiment, the processor/controller 402 may drive the main servo/actuator 444 and medicine door motor 446 to position and expose the active compartment. In a node-based embodiment, the processor/controller 402 may operate a digital lock, motor driver, LED subsystem, reed switch, and weight sensor associated with a storage drawer or node unit. The processor/controller 402 may then receive sensor feedback, generate event data, store event data in the local logs/cache 432, and transmit event data to the network/backend 434.

The architecture 400 therefore provides a common electronic foundation for multiple medication storage implementations, including the carousel embodiment of FIG. 3A and the node-based embodiment of FIG. 3B.

### Prescription Extraction Pipeline

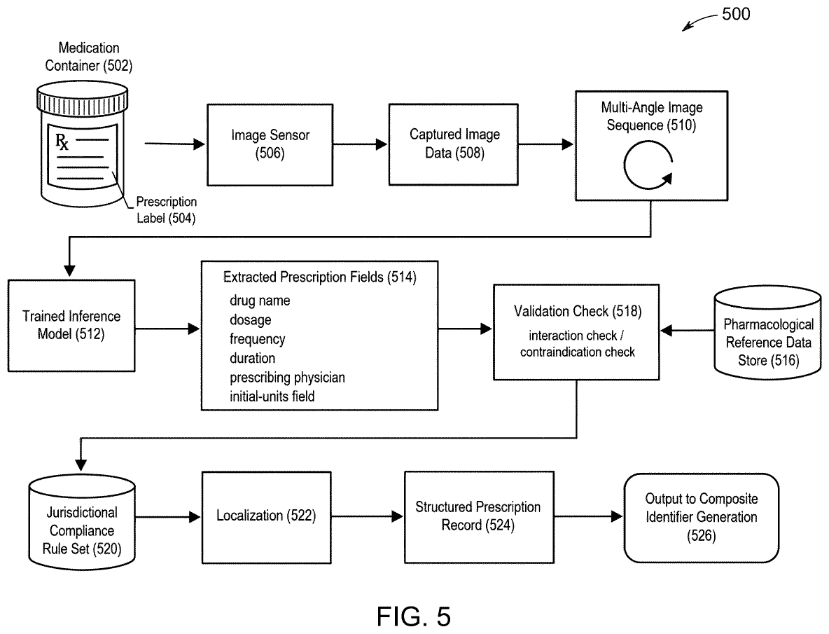

FIG. 5 illustrates a prescription extraction pipeline 500 for generating a structured prescription record from prescription label image data, in accordance with an embodiment. The prescription extraction pipeline 500 may implement the prescription information module described for the medication adherence system. The pipeline 500 converts prescription-level information into structured data that may be used for composite identifier generation, medication scheduling, placement verification, dispensing control, medication validation, adherence tracking, and wellness-score computation.

The prescription extraction pipeline 500 begins with a medication container 502 carrying a prescription label 504. The medication container 502 may include a standard pharmacy bottle, vial, blister pack, cartridge, pouch, inhaler container, or other medication container. The prescription label 504 may include printed text, handwritten text, pharmacy-generated information, physician-generated information, dosage instructions, medication identifiers, refill information, machine-readable codes, or other prescription-related information.

An image sensor 506 captures image data of the prescription label 504. The image sensor 506 may be part of the user client device 104, the medication dispensing apparatus 130, a dedicated scanning device, a controller unit, or another image-capture device. The captured image data 508 may include a single image, multiple still images, a burst sequence, a video sequence, or other image data sufficient to capture prescription information.

In some implementations, the captured image data 508 is organized as a multi-angle image sequence 510. The multi-angle image sequence 510 may be captured while the medication container 502 is rotated relative to the image sensor 506, while the image sensor 506 moves relative to the medication container 502, or while the user captures multiple views of the prescription label 504. The rotation may be clockwise, counter-clockwise, partial, continuous, manual, motorized, or otherwise controlled. This supports scenarios in which the prescription label wraps around a medication container and cannot be captured completely from a single viewing angle.

The multi-angle image sequence 510 is provided to a trained inference model 512. The trained inference model 512 may include an OCR model, computer-vision model, image-processing model, language model, named-entity-recognition model, field-classification model, rule-based parser, statistical model, machine-learning model, or hybrid model. The trained inference model 512 may process printed labels, handwritten prescriptions, scanned prescriptions, pharmacy labels, doctor instructions, or uploaded prescription images. The trained inference model 512 may also produce confidence values or flags for user, caregiver, provider, or pharmacist review when the extracted data does not satisfy a threshold confidence level.

The trained inference model 512 generates extracted prescription fields 514. The extracted prescription fields 514 may include drug name, dosage, frequency, timing, duration, prescription identifier, prescribing physician, pharmacy identifier, refill information, initial-units field, special instructions, medication identifier, or additional metadata. In some implementations, extracted fields may include both user-facing medication instructions and machine-readable values usable by the backend system 108 for scheduling, verification, and dispensing control.

The pharmacological reference data store 516 provides reference information for validation. The validation check 518 may compare the extracted prescription fields 514 against the pharmacological reference data store 516. The validation check 518 may verify medication names, dosage ranges, frequencies, interaction conditions, contraindication conditions, allergy-related warnings, regimen consistency, or other prescription-safety information. The validation check 518 may identify missing fields, inconsistent fields, low-confidence fields, or values requiring manual review.

The jurisdictional compliance rule set 520 may provide region-specific or organization-specific rules for formatting, presentation, units, language, medication naming, schedule format, instruction format, or prescription record completeness. The localization 522 may adapt the validated prescription fields into a region-appropriate or user-appropriate format. The localization 522 may convert units, format timing instructions, normalize medication names, translate instructions, apply local date formats, or otherwise prepare the extracted prescription data for downstream use.

The structured prescription record 524 is generated from the validated and localized prescription fields. The structured prescription record 524 may be stored in the backend system 108, associated with the user profile record, associated with the medication container 502, associated with a medication identifier, or linked to a placement record. The structured prescription record 524 may include fields used to schedule dispensing events, determine expected consumed units, compute refill timing, generate reminder states, validate medication placement, and support health-score computation.

The structured prescription record 524 is provided as output to composite identifier generation 526. The composite identifier generation 526 may use the structured prescription record 524 together with a user profile record, device identifier, and placement record to generate a composite identifier. In this way, the prescription information extracted from the prescription label 504 is not isolated from the later dispensing process. Instead, it becomes part of the data context that controls placement verification, control-instruction generation, sensor-based verification, event-record storage, and medication adherence reporting.

### Method of Operating the Medication Dispensing Apparatus

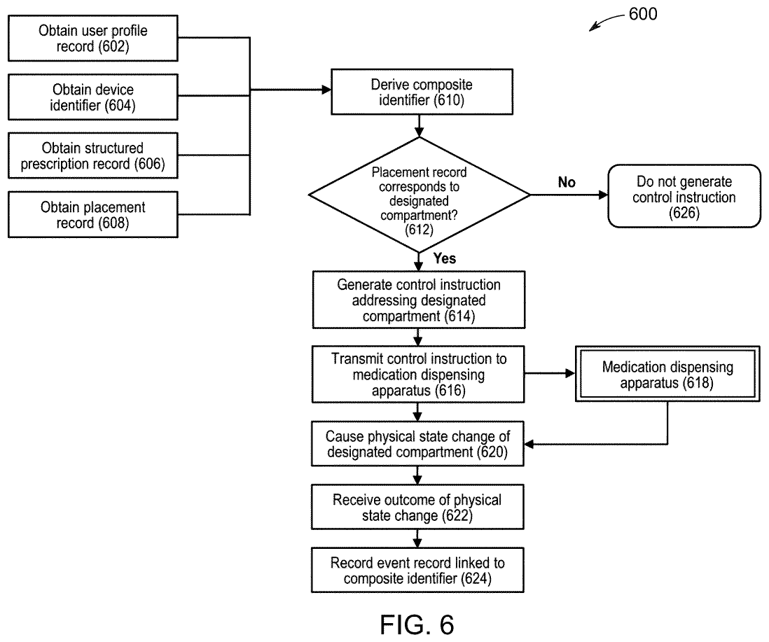

FIG. 6 illustrates a method 600 of operating the medication dispensing apparatus 130 of FIG. 1 to carry out a dispensing cycle under the composite identifier of FIG. 2. The method 600 derives a composite identifier from a plurality of input records, conditionally generates and transmits a control instruction addressing a designated compartment, causes a physical state change of the designated compartment, receives an outcome of the physical state change, and records an event record linked to the composite identifier on a non-transitory storage medium. The method 600 may be performed by the one or more processors 110 of the backend system 108 executing instructions stored on the one or more non-transitory storage media 112. In other implementations, one or more steps of the method 600 may be performed by the medication dispensing apparatus 130, the medication dispensing apparatus 300 of FIG. 3A, the node-based medication storage unit 372 of FIG. 3B, the user client device 104, or a distributed combination thereof. The method 600 may be triggered by a scheduled dispensing time maintained by the temporal state management 224 of FIG. 2, by a manual dispensing request received through the user client device 104, by a caregiver-authorized request, or by a command received from a healthcare provider device 136.

At step 602, the method 600 obtains the user profile record. The user profile record may be retrieved from a database maintained by the backend system 108, received from a user-facing application on the user client device 104, obtained from a healthcare record system, or generated during onboarding of the user. The user profile record may include demographic information, medical history, allergy information, baseline biometric information, caregiver information, emergency-contact information, medication-regimen preferences, or other health-profile information associated with the user.

At step 604, the method 600 obtains the device identifier of the peripheral data source 106 paired with the user. The device identifier may identify a wearable device, medical monitoring device, fitness device, user client device, controller unit, node unit, medication dispensing apparatus, or other peripheral data source associated with the user. The device identifier may be obtained by scanning a machine-readable code through the image sensor 140, receiving the device identifier through a user interface, manual entry, wireless discovery, NFC interaction, RFID reading, Bluetooth pairing, Wi-Fi discovery, or another device-onboarding process.

At step 606, the method 600 obtains the structured prescription record for the medication associated with the medication container. Consistent with FIG. 5, the structured prescription record may be obtained by applying the trained inference model 118 to image data of a prescription label, including a multi-angle image sequence in some implementations. The structured prescription record may be validated against the pharmacological reference data store 120 for medication-name validation, dosage validation, drug-interaction information, contraindication information, or other medication-safety information. The structured prescription record may also be localized into a region-appropriate or user-appropriate format using the jurisdictional compliance rule set 124.

At step 608, the method 600 obtains the placement record indicating assignment of the medication container to the designated compartment. The designated compartment may be a carousel compartment, drawer, bay, bin, slot, vial receptacle, node-based compartment, or other medication-container receiving region. The placement record may be generated from a user input event at the display/touchscreen 304 of FIG. 3A, from a user client device 104, from a caregiver input, from a controller interface 388 of FIG. 3B, from a read of an RFID tag or NFC tag, from a machine-readable code, from image recognition performed using the image sensor 140, or from another placement-confirmation process. In a carousel-based embodiment, the placement record may be cross-referenced against an installed compartment count communicated by the configuration-encoding interface 314 of FIG. 3A. In a node-based embodiment, the placement record may be cross-referenced against a node identifier, drawer identifier, or storage-compartment identifier associated with the node-based medication storage unit 372 of FIG. 3B.

At step 610, the method 600 derives the composite identifier from the user profile record, the device identifier, the structured prescription record, and the placement record. The composite identifier may implement, extend, or operate together with a Health Record Identifier or unique digital record associated with the user. The composite identifier may be implemented as a structured tuple, database key, linked record, generated identifier, secure identifier, or another data structure that associates the user context, device context, prescription context, and medication-placement context. In some implementations, the composite identifier may be generated from all four records. In other implementations, the composite identifier may be generated based at least in part on one or more of those records and may further reference a medication identifier, compartment identifier, node identifier, storage-device identifier, regimen identifier, schedule identifier, or event identifier.

At decision step 612, the method 600 evaluates whether the placement record corresponds to the designated compartment addressed for the dispensing operation. The evaluation may compare the designated compartment indicated by the placement record with the designated compartment to be addressed by the control instruction. The evaluation may also verify that the medication container corresponds to the structured prescription record, that the user remains associated with the peripheral data source 106, that the medication container has not been removed or reassigned since the placement record was generated, and that the scheduled dispensing event corresponds to the medication regimen. The evaluation implements the placement-compartment correspondence check 214 of FIG. 2 and gates generation or transmission of the control instruction.

When the evaluation returns a negative result, the method 600 proceeds to step 626 and inhibits generation or transmission of the control instruction. The method 600 may also record an exception event record in the event record store 126, present a message on the user client device 104 or the display/touchscreen 304, request that the user re-verify the medication placement, request caregiver confirmation, or transmit a notification 154 indicating that the placement record does not match the intended dispensing operation.

When the evaluation returns an affirmative result, the method 600 proceeds to step 614 and generates the control instruction addressing the designated compartment. The control instruction may include an instruction code identifying the dispensing operation, an identifier of the designated compartment, the composite identifier, a timestamp, a schedule identifier, a medication identifier, a storage-device identifier, or one or more device-control parameters. In a carousel-based implementation, the control instruction may specify a rotational position or indexing operation for the rotatable storage member/carousel 322 of FIG. 3A and an access operation for the movable access member/medicine door 338. In a node-based implementation, the control instruction may specify an unlocking, opening, extension, illumination, or access-control operation for the medication drawer/storage compartment 374 of FIG. 3B.

At step 616, the method 600 transmits the control instruction to the medication dispensing apparatus 618 through the communications interface 114 of FIG. 1. The transmission may occur through a wired communication path, wireless communication path, local-area network, wide-area network, cellular link, Bluetooth link, Wi-Fi link, USB connection, Ethernet connection, or another communication pathway. In some implementations, the transmission may be secured using encryption, authentication, access-control, session validation, device-pairing validation, or another data-protection technique.

At step 618, the medication dispensing apparatus receives the control instruction. The medication dispensing apparatus may be the medication dispensing apparatus 130 of FIG. 1, the carousel-based medication dispensing apparatus 300 of FIG. 3A, the node-based medication storage unit 372 of FIG. 3B, or another controlled-access medication storage device. The medication dispensing apparatus may verify that the addressed compartment, drawer, node, bay, or storage region is in a known state and may confirm readiness to perform the commanded physical state change.

At step 620, the method 600 causes the physical state change of the designated compartment. In the carousel-based implementation of FIG. 3A, the controller 302 may command the actuator 334 to move the rotatable storage member/carousel 322 through the coupler assembly 336 so that the designated compartment, such as the active second compartment 326, is positioned at the dispensing zone/output 342. The controller 302 may further cause the movable access member/medicine door 338 to expose the designated compartment while restricting access to other compartments. The lighting floor/LED illumination 340 may provide visual feedback associated with the active compartment or dispensing state. In the node-based implementation of FIG. 3B, the controller interface 388 may actuate the digital lock/smartlock 378 and the motor driver/actuator 380 to unlock, open, extend, or otherwise provide access to the medication drawer/storage compartment 374. Thus, the physical state change may include rotating, indexing, unlocking, opening, exposing, extending, illuminating, or otherwise presenting the designated compartment.

At step 622, the method 600 receives an outcome of the physical state change from the medication dispensing apparatus. In a carousel-based implementation, the outcome may include an actuator-status signal from the actuator 334, a position signal indicating that the rotatable storage member/carousel 322 reached the intended position, a door-state signal associated with the movable access member/medicine door 338, a weight reading from the weight sensor/load cell 312, an input received through the display/touchscreen 304, a microphone input received through the microphone 308, or an output-state indication associated with the lighting floor/LED illumination 340. In a node-based implementation, the outcome may include a lock-state signal from the digital lock/smartlock 378, a motor-state signal from the motor driver/actuator 380, an open/closed or extension-state signal from the reed switch/position sensor 382, a weight reading from the weight sensor/load cell 384, an LED state of the LED subsystem 386, or event data/sensor data output 392. The outcome may further include an error indicator, timeout indication, unexpected-position indication, sensor-failure indication, or user-confirmation status.

At step 624, the method 600 records the event record linked to the composite identifier on the non-transitory storage medium. The event record may include the composite identifier, a timestamp, a medication identifier, a prescription identifier, an identifier of the designated compartment, a node identifier, a drawer identifier, a storage-device identifier, the outcome of the physical state change, one or more sensor readings, a confirmation status, reminder status, access status, error status, or adherence status. The event record may be stored in the event record store 126, in local storage of the medication dispensing apparatus, on the user client device 104, in backend storage, or across distributed storage resources.

In some implementations, the event record may be encrypted, access-controlled, synchronized, backed up, secured, or integrity-protected. For example, a data-protection operation may be applied to at least a portion of the event record, the composite identifier, or both. Such integrity protection may include a cryptographic operation in some embodiments, but the method 600 may not implement cryptographic audit binding in every implementation. The stored event record may later be used for adherence review, caregiver notification, healthcare-provider reporting, refill forecasting, wellness-score updates, subsequent dispensing control, or anomaly analysis.

Method that Generates a Verification Result for a Dispensing Operation

FIG. 7 illustrates a method 700 that generates a verification result for a dispensing operation performed by the method 600 of FIG. 6 and that modifies a subsequent dispensing operation directed to the designated compartment when a discrepancy exceeding a configurable tolerance value is detected. The method 700 implements the weight-based verification 220 of FIG. 2 and is performed by the one or more processors 110 executing instructions stored on the non-transitory storage medium 112. The method 700 may be triggered upon completion of the dispensing operation, at a scheduled audit time, or in response to a request received from a healthcare provider device 136 or a caregiver device 134.

At step 706, the method 700 receives a baseline container weight measurement from a weight sensor/load cell 704 of the medication dispensing apparatus 702, which implements the weight sensor/load cell of FIG. 3A, the weight sensor/load cell 384 of FIG. 3B, or another weight sensor associated with the medication dispensing apparatus. The baseline container weight measurement is captured prior to the dispensing operation and represents the total weight of the medication container together with the medication contained therein.

At step 708, the method 700 receives a current container weight measurement from the weight sensor/load cell 704, captured after the dispensing operation and representing the total weight of the medication container after a portion of the medication has been removed by the user. In some implementations, capture of the current container weight measurement is timed by a stable-weight detection routine that identifies when the medication container has been returned to the designated compartment and the weight reading has settled within a predetermined variation range, and may apply compensations for ambient temperature, vibration, or tilt of the medication dispensing apparatus 702.

At step 710, the method 700 computes a weight differential as:

ΔW=W_{baseline}−W_{current}
where ΔW is the weight differential, W_{baseline} is the baseline container weight measurement, and W_{current} is the current container weight measurement.

At step 712, the method 700 retrieves or determines a per-unit-mass value associated with the medication. In some implementations, the per-unit-mass value is obtained from a unit-mass field of the structured prescription record, from a reference data store of medication unit masses indexed by a national drug code (NDC) or another medication identifier, or by empirical measurement performed at the time the medication container is loaded. The per-unit-mass value may be a single value for medications with uniform unit masses (e.g., compressed tablets), or a range value for medications with non-uniform unit masses (e.g., gel capsules or coated tablets).

At step 714, the method 700 computes a consumed-units quantity from the weight differential and the per-unit-mass value as:

N_{consumed}=ΔW/mᵤₙᵢₜ
where N_{consumed} is the consumed-units quantity and mᵤₙᵢₜ is the per-unit-mass value. The consumed-units quantity may be rounded to a nearest integer, may be expressed as a real number to capture partial-dose removal, or may be capped at a predetermined maximum to suppress unrealistic readings attributable to sensor anomalies or tampering.

At step 716, the method 700 determines an initial-units quantity according to a predetermined precedence order applied across three sources.

At step 718, the method 700 examines source (i), the initial-units field of the structured prescription record. When the initial-units field is populated, for example, where the prescribing physician or pharmacy has indicated the original tablet count, the initial-units quantity is taken from the initial-units field and the method 700 proceeds to step 724.

At step 720, when the initial-units field is not populated, the method 700 examines source (ii), a reference empty-container weight retrieved from the empty-container weight data store 122 of FIG. 1 using a container category of the medication container. The container category may be one of thirty-dram, sixteen-dram, eight-dram, or another industry-standard container size. Where source (ii) is applied, the initial-units quantity is computed as:

Nᵢₙᵢₜᵢₐₗ=(W_{baseline}−W_{empty_ref})/mᵤₙᵢₜ
where W_{empty_ref} is the reference empty-container weight.

At step 722, when neither source (i) nor source (ii) yields a value (for example, when the empty-container weight data store does not contain an entry for the container category), the method 700 applies source (iii), a default value. In one example, the default value is one hundred units.

At step 724, the method 700 computes a remaining-units quantity as:

N_{remaining}=Nᵢₙᵢₜᵢₐₗ−N_{consumed}

At step 726, the method 700 derives an expected-units quantity from the structured prescription record. The expected-units quantity represents the number of medication units scheduled to be consumed during the dispensing operation according to the dosage and frequency fields of the structured prescription record, and may be a single unit, multiple units, or a fractional value.

At step 728, the method 700 compares the consumed-units quantity to the expected-units quantity. At step 730, the method 700 generates a verification result based on the comparison. The verification result may indicate one of a successful dispensing event (when the consumed-units quantity matches the expected-units quantity within a tolerance), an under-consumption event (consumed-units quantity less than the expected-units quantity by more than the tolerance), an over-consumption event (consumed-units quantity greater than the expected-units quantity by more than the tolerance), or a tampering event (for example, where the consumed-units quantity exceeds a predetermined threshold or the weight differential exhibits a non-monotonic profile inconsistent with normal medication removal).

At decision step 732, the method 700 evaluates whether the discrepancy indicated by the verification result exceeds a configurable tolerance value stored on the non-transitory storage medium. The configurable tolerance value may be configured by a healthcare provider, by a caregiver, or by a system administrator; may be applied globally to all medications or stored as a per-medication entry (e.g., on the structured prescription record or in a reference data store); and may be expressed as an absolute number of units, a percentage of the expected-units quantity, or a probabilistic threshold derived from historical adherence data of the user.

When the evaluation at decision step 732 returns a negative result, the method 700 continues, at step 742, the subsequent dispensing operation according to the established schedule, and in some implementations logs a successful-verification entry in the event record store 126 and transmits a positive adherence indicator to the caregiver device 134 or healthcare provider device 136.

When the evaluation at decision step 732 returns a positive result, the method 700 modifies, at step 734, the subsequent dispensing operation directed to the designated compartment through a subsequent control instruction generated by the control instruction generation 216 of FIG. 2. The modification comprises at least one of:

halting the subsequent dispensing operation at step 736, such that the medication dispensing apparatus 702 is prevented from causing a further physical state change until a manual override is received;

delaying the subsequent dispensing operation at step 738 by a configurable delay interval ranging from a few minutes to several hours depending on the severity of the discrepancy; or
requiring an additional authorization at step 740, such as a confirmation signal from a healthcare provider device 136 transmitted through the communications interface, a one-time passcode entered by the user, or a biometric verification of the user.

In this manner, the method 700 closes the loop between the sensor-derived verification result and subsequent control instructions generated by the control instruction generation 216 of FIG. 2, so that physically observed consumption behaviour directly constrains subsequent physical state changes of the designated compartment.

### Wellness Score Computation Pipeline

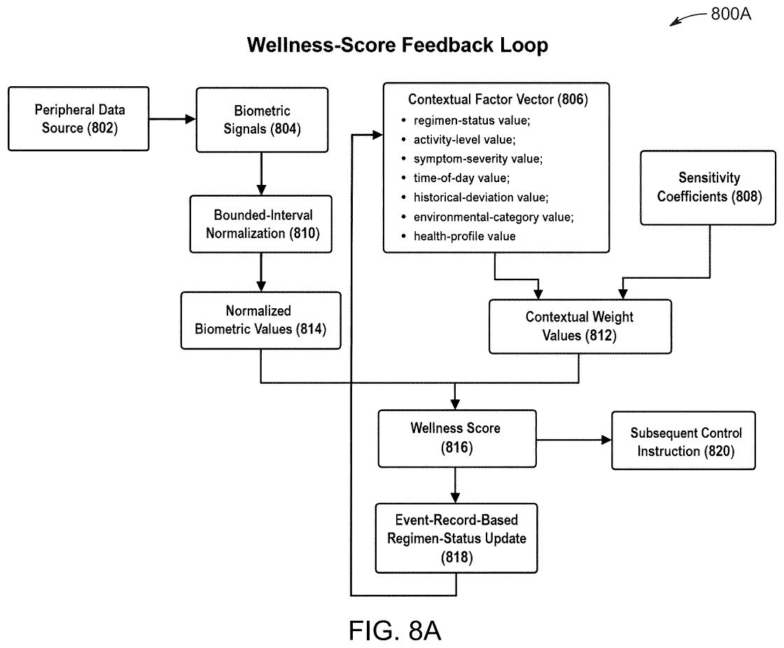

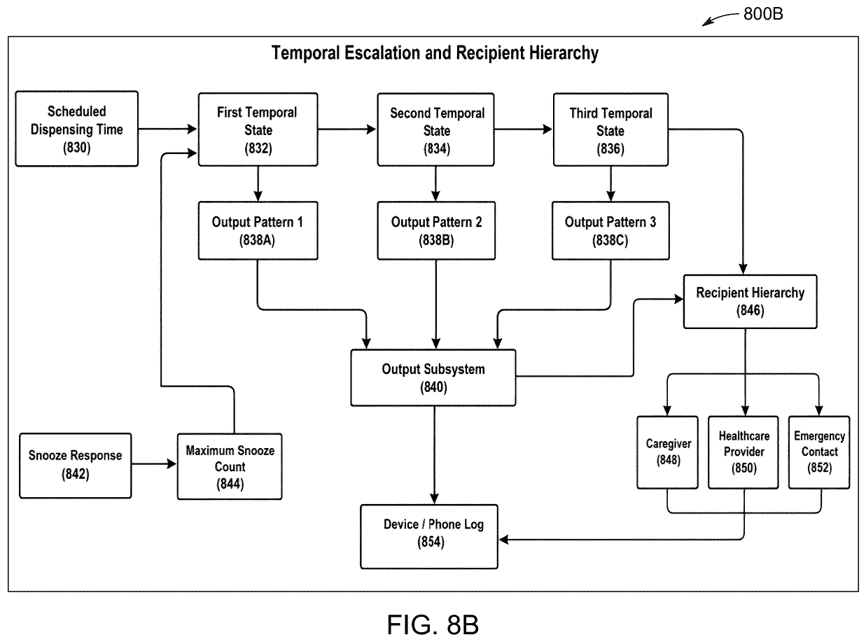

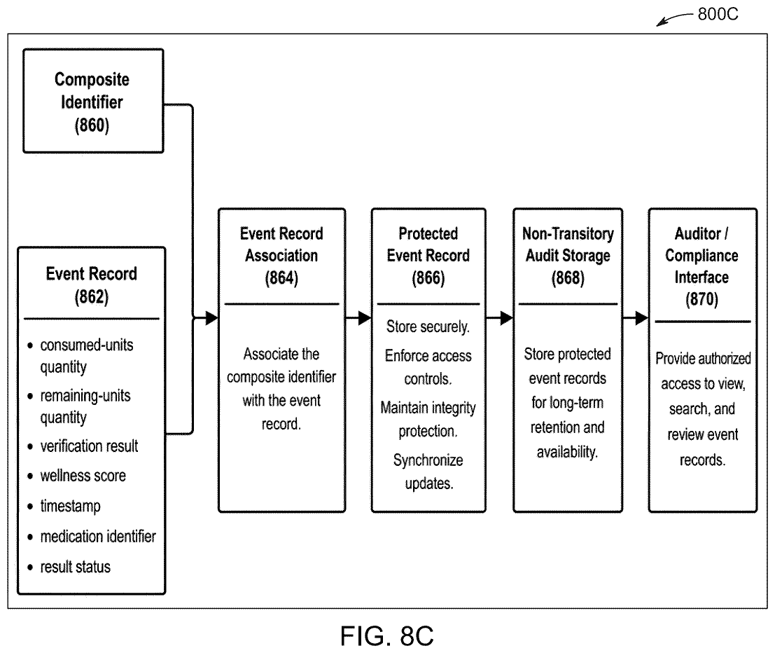

FIG. 8A illustrates a wellness score computation pipeline 800A that computes a wellness score 816 for the user from a plurality of biometric signals 804 received from the peripheral data source 802, and provides the wellness score 816 as an input to a subsequent control instruction 820 directed to the designated compartment of the medication dispensing apparatus. The pipeline 800A implements the wellness score computation 222 of FIG. 2 and is performed by the one or more processors 110 executing instructions stored on the non-transitory storage medium 112. The pipeline 800A operates in coordination with the method 600 of FIG. 6 and the method 700 of FIG. 7, and consumes the regimen-status value updated from event records recorded under the composite identifier.

The peripheral data source 802 corresponds to the peripheral data source 106 of FIG. 1, and may communicate the plurality of biometric signals 804 through the wireless interface 116 (e.g., over BLE, Wi-Fi, NFC, or a cellular standard). The plurality of biometric signals 804 may include one or more of a heart-rate (HR) signal, a heart-rate variability (HRV) signal, a blood-oxygen saturation (SpO2) signal, a skin-temperature signal, a galvanic-skin-response signal, an activity signal (e.g., steps or accelerometer-derived motion intensity), a sleep signal (e.g., sleep stages and duration), a blood-pressure signal, a blood-glucose signal, an ECG signal, or an EEG signal. The plurality of biometric signals 804 may be sampled continuously, periodically, or upon a triggered event, and may be transmitted in real time, in periodic batches, or upon a synchronization event with the system.

The pipeline 800A constructs a contextual factor vector 806 for each biometric signal of the plurality of biometric signals 804. In some implementations, the contextual factor vector 806 comprises values for one or more of a regimen-status value, an activity-level value, a symptom-severity value, a time-of-day value, a historical-deviation value, an environmental-category value, or a health-profile value. The regimen-status value reflects the user's recent adherence to the medication schedule and may be expressed as a normalized score derived from prior dispensing events recorded under the composite identifier of the user. The activity-level value may be derived from accelerometer, gyroscope, or step-count data of the peripheral data source 802. The symptom-severity value may be derived from a symptom-diary application, a clinical questionnaire, or anomaly-detection signals applied to the plurality of biometric signals 804. The time-of-day value may be encoded as a circadian-cycle position. The historical-deviation value reflects a deviation of the biometric signal from a historical baseline of the user. The environmental-category value reflects ambient temperature, humidity, altitude, or air-quality index. The health-profile value reflects underlying health conditions, comorbidities, age group, sex, or other clinical factors associated with the user.

The pipeline 800A retrieves a set of sensitivity coefficients 808 from the non-transitory storage medium. In some implementations, the set of sensitivity coefficients 808 comprises a vector of weights w=[w₁, w₂, . . . , wₙ], with each weight corresponding to a contextual factor of the contextual factor vector 806. The set of sensitivity coefficients 808 may be defined by a healthcare provider, may be derived from a population-level model trained on aggregated clinical data, or may be personalized to the user through an adaptive routine that updates the weights based on observed adherence outcomes and biometric trends.

At step 810, the pipeline 800A applies a bounded-interval normalization to each biometric signal of the plurality of biometric signals 804 to produce a normalized biometric value 814. In one example, the bounded-interval normalization is a min-max normalization expressed as:

x_{normalized}=(x−xₘᵢₙ)/(xₘₐₓ−xₘᵢₙ)
such that x_{normalized} falls within the interval [0, 1]. The values of xₘᵢₙ and xₘₐₓ may be set per biometric-signal type based on physiologically meaningful bounds, may be derived from a historical range of the user, or may be derived from a population-level distribution.

At step 812, the pipeline 800A produces a contextual weight value for each biometric signal by combining the contextual factor vector 806 with the set of sensitivity coefficients 808. In one example, the contextual weight value for the i-th biometric signal is:

$${W}_{i} = {\sum }_{j} ( {w}_{j} \times {c}_{j , i} )$$

where wⱼ is the j-th sensitivity coefficient and c_{j,i} is the j-th contextual factor value for the i-th biometric signal.

At step 816, the pipeline 800A aggregates products of the contextual weight values and the corresponding normalized biometric values across the plurality of biometric signals 804 to produce the wellness score 816, expressed in one example as:

$$S = {\sum }_{i} ( {W}_{i} \times {x}_{normalized , i} )$$

The wellness score 816 may further be bounded to a predetermined range (e.g., 0≤S≤100) by a final scaling step that maps the raw aggregate to the predetermined range.

At step 818, the pipeline 800A updates the regimen-status value of the contextual factor vector 806 from a most recent event record recorded under the composite identifier, consistent with the feedback path. The update reflects the most recent adherence outcome of the user, such as a successful dispensing event, an under-consumption event, an over-consumption event, a missed dispensing event, or a tampering event, so that a subsequent computation of the wellness score 816 reflects the most recent event record.

The wellness score 816 is provided to the control instruction generation 216 of FIG. 2, such that the subsequent control instruction 820 directed to the designated compartment of the medication dispensing apparatus is based at least in part on the wellness score 816. When the wellness score 816 is consistent with proceeding with the next scheduled dispensing operation, the subsequent control instruction 820 addresses the designated compartment as scheduled. When the wellness score 816 indicates a deviation that warrants schedule modification, the subsequent control instruction 820 may halt, delay, or otherwise modify the next scheduled dispensing operation, and may trigger a notification 154 to a caregiver device 134 or healthcare provider device 136 of the recipient hierarchy described in FIG. 1.

### Temporal State Management Pipeline

FIG. 8B illustrates a temporal state management pipeline 800B that drives the output subsystem 840 of the medication dispensing apparatus through a sequence of output patterns according to a current temporal state of a scheduled dispensing event, and that transmits a notification to a recipient of a recipient hierarchy 846 distinct from the user when a defined temporal state of the sequence is reached. The pipeline 800B implements the temporal state management 224 of FIG. 2 and is performed by the one or more processors 110 executing instructions stored on the non-transitory storage medium 112. The pipeline 800B operates in coordination with the method 600 of FIG. 6 and the method 700 of FIG. 7 to manage user-facing alerts associated with each scheduled dispensing event.

The pipeline 800B receives a scheduled dispensing time 830 (Ts) for the medication. The scheduled dispensing time 830 may be derived from the frequency and timing fields of the structured prescription record associated with the medication, and may be a specific clock time, a relative offset from another scheduled event, or a context-dependent time defined by physiological signals (e.g., wake-up time or post-meal time).

The pipeline 800B defines, for the scheduled dispensing event, a sequence of temporal states comprising at least a first temporal state 832, a second temporal state 834, and a third temporal state 836, ordered by increasing elapsed time from the scheduled dispensing time 830. In some implementations, the first temporal state 832 corresponds to an initial window extending from Ts to a first time threshold T₁; the second temporal state 834 corresponds to a reminder window extending from T₁ to a second time threshold T₂; and the third temporal state 836 corresponds to an escalation window extending from T₂ to a third time threshold T₃. In one example, T₁ is approximately ten minutes, T₂ is approximately thirty minutes, and T₃ is approximately sixty minutes after the scheduled dispensing time 830. The values of T₁, T₂, and T₃ may be configured by a healthcare provider, set as default values, or personalized to the user based on historical adherence patterns.

Each temporal state of the sequence is associated with a distinct output pattern that the pipeline 800B drives through the output subsystem 840 of the medication dispensing apparatus. The first temporal state 832 is associated with a first output pattern 838A comprising, in some implementations, a soft reminder that includes one or more of a visual indicator on the display/touchscreen, an audio indicator through the speaker, an LED indication through the lighting floor/LED illumination, and an on-screen prompt. The second temporal state 834 is associated with a second output pattern 838B comprising, in some implementations, a persistent reminder that includes one or more of a more pronounced visual indicator, a louder audio indicator, a haptic indicator, an LED indication, an on-screen prompt, and a push notification transmitted to the user client device 104. The third temporal state 836 is associated with a third output pattern 838C comprising, in some implementations, an escalation alert that includes one or more of an emphatic visual indicator, an emphatic audio indicator, a haptic indicator, an LED indication, an on-screen prompt, and an escalation message transmitted through the communications interface to the recipient hierarchy 846.

The output subsystem 840 of the medication dispensing apparatus may include a visual display, an audio speaker, a haptic actuator, an LED indicator, a screen-based message interface, and a network interface, and receives the output patterns 838A, 838B, 838C from the pipeline 800B and renders each output pattern through the corresponding output channel.

The pipeline 800B may receive a snooze response 842 from the user during the first temporal state 832 or the second temporal state 834, as a touch input on the display/touchscreen, a voice command captured by the microphone, a button press on the medication dispensing apparatus, or a remote command transmitted from the user client device 104. In response to receiving the snooze response 842, the pipeline 800B resets the sequence of temporal states to the first temporal state 832 and increments a maximum-snooze counter 844. The maximum-snooze counter 844 tracks the number of consecutive snooze resets performed by the user for the scheduled dispensing event and is bounded by a predetermined maximum Nₘₐₓ such that 0≤n≤Nₘₐₓ. In one example, Nₘₐₓ is five.

When the maximum-snooze counter 844 has reached Nₘₐₓ, the pipeline 800B no longer accepts further snooze responses 842 and continues the temporal state progression. Upon entry into the third temporal state 836, either through normal time progression or through exhaustion of Nₘₐₓ, the pipeline 800B transmits a notification to a notification recipient of the recipient hierarchy 846 comprising at least one recipient distinct from the user.

The recipient hierarchy 846 corresponds to the recipient devices 132 of FIG. 1 and may include a caregiver 848, a healthcare provider 850, and an emergency contact 852. The recipient hierarchy 846 may be configured with priority ordering and severity thresholds, such that lower-severity adherence events trigger notifications to lower-priority recipients and higher-severity adherence events trigger notifications to further recipients including the emergency contact 852.

The pipeline 800B may further log each output-pattern transition, each snooze response 842, and each escalation notification in a device/phone log 854. The device/phone log 854 may be maintained on the medication dispensing apparatus, on the user client device 104, or on the backend system 108, and may be synchronized with the event record store 126 associated with the composite identifier of the user, providing a complete record of temporal-state management activity for compliance audit and clinical review.

### Event Record Association, Protected Storage, and Audit Access

FIG. 8C illustrates an event-record association, protected-storage, and audit-access workflow, in accordance with an embodiment. FIG. 8C shows how an event record generated by the system may be associated with a composite identifier, converted or maintained as a protected event record, stored in non-transitory audit storage, and made available through an authorized auditor or compliance interface. The workflow supports traceable event review without requiring cryptographic audit binding.

A composite identifier 860 and an event record 862 are provided to an event record association 864. The composite identifier 860 may correspond to the composite identifier 212 of FIG. 2 and may identify a medication-specific operating context of the user. In some implementations, the composite identifier 860 may be generated based at least in part on a user profile record, a device identifier, a structured prescription record, and a placement record. In other implementations, the composite identifier 860 may implement, extend, or operate together with a Health Record Identifier, unique digital record, medication-context identifier, or other record association that links the user, device, prescription, and medication-storage context.

The event record 862 may represent a dispensing event, attempted dispensing event, placement event, verification event, reminder event, snooze event, escalation event, wellness-score update, medication-loading event, device-pairing event, or another event generated by the system. The event record 862 may include one or more record fields, such as a consumed-units quantity, remaining-units quantity, verification result, wellness score, timestamp, medication identifier, result status, compartment identifier, node identifier, drawer identifier, storage-device identifier, user-confirmation status, reminder status, sensor reading, or physical state-change outcome. The result status may indicate, for example, successful dispensing, verification failure, missed dispensing, delayed confirmation, access failure, low medication level, unexpected removal, or tamper indication.

The event record association 864 associates the composite identifier 860 with the event record 862. The association may be implemented by storing the composite identifier 860 as a field in the event record 862, storing a pointer or relational link between the composite identifier 860 and the event record 862, indexing the event record 862 by the composite identifier 860, linking the event record 862 to a user profile record, device record, prescription record, placement record, medication regimen record, or storing the event record 862 in a database table, event log, object store, or another data structure associated with the composite identifier 860. In this manner, the event record 862 remains traceable to the relevant user, device, medication, prescription, compartment, and placement context.

The event record association 864 may produce or update a protected event record 866. The protected event record 866 may be stored securely, access-controlled, synchronized, backed up, versioned, retained, or integrity-protected using one or more data-protection techniques. In some implementations, the protected event record 866 may be protected by authentication, authorization, role-based access control, encryption at rest, encryption in transit, secure synchronization, redundant storage, database-level protection, checksum-based integrity checking, backup policies, retention policies, or administrative access restrictions. The protected event record 866 does not require a cryptographic binding to the composite identifier in every implementation.

The protected event record 866 is stored in non-transitory audit storage 868. The non-transitory audit storage 868 may include local storage, backend storage, cloud storage, storage on the medication dispensing apparatus, storage on the user client device, caregiver-accessible storage, healthcare-provider-accessible storage, or distributed storage. The non-transitory audit storage 868 may retain event records for adherence review, caregiver review, healthcare-provider review, refill forecasting, wellness-score updates, anomaly analysis, technical support, organizational review, or long-term record availability.

An auditor/compliance interface 870 provides authorized access to stored event records. The auditor/compliance interface 870 may allow an authorized user, caregiver, healthcare provider, administrator, support representative, reviewer, or other authorized entity to view, search, filter, retrieve, export, or review event records. The auditor/compliance interface 870 may present records by user, medication, prescription, event type, date range, compartment, node, drawer, storage device, result status, wellness score, verification result, reminder escalation, or other record field. Access to the auditor/compliance interface 870 may be controlled based on user credentials, role-based permissions, caregiver authorization, provider authorization, administrator approval, or another access-control policy.

In an example operation, a dispensing event produces the event record 862. The event record association 864 links the event record 862 with the composite identifier 860 so that the event record remains associated with the correct user, device, prescription, and medication placement. The system then stores the associated record as the protected event record 866 using secure storage, access-control, synchronization, and retention techniques. The protected event record 866 is retained in the non-transitory audit storage 868. Later, an authorized reviewer may access the record through the auditor/compliance interface 870 to review medication adherence, confirm a dispensing outcome, evaluate a missed-dose escalation, inspect a weight-verification result, review a wellness-score update, or confirm that a medication event was recorded.

FIG. 8C therefore supports traceable recordkeeping and authorized review while keeping the core technology focused on composite-identifier-based association, protected event-record storage, controlled access to records, and medication-adherence auditing. The workflow may be used with the carousel-based medication dispensing apparatus of FIG. 3A, the node-based medication storage unit of FIG. 3B, or another controlled-access medication storage implementation.

### Artificial Intelligence System Implementation

The foregoing disclosure establishes an operational loop in which a composite identifier, derived from a user profile record, a device identifier of a paired peripheral data source, a structured prescription record, and a placement record, anchors each control instruction, physical state change, verification result, wellness score, and protected event record produced by the system. Within that loop, prescription understanding, contextual health scoring, and consumption verification are expressed as computational operations performed by the one or more processors on inputs bound to the composite identifier. The implementations described hereafter realize those operations as a coordinated artificial-intelligence (AI) implementation architecture comprising a plurality of models, inference runtimes, training pipelines, and model-governance components that cooperate under the same composite identifier to produce the structured prescription record, the wellness score, the verification result, and the subsequent control instruction consumed by the operational loop. The following figures accordingly describe this AI implementation architecture and its integration points with the control instruction generation, the event record store, and the recipient hierarchy already described.

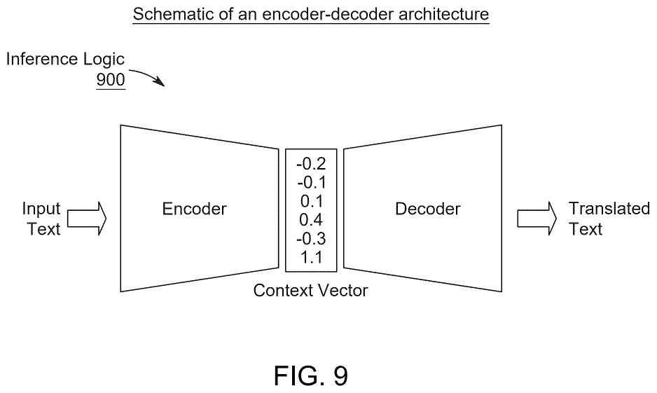

The inference logic 900 operative within the artificial-intelligence components of the medication adherence system, is described in further detail with reference to FIG. 9 through FIG. 19.

Some implementations of the technology disclosed relate to using a Transformer model to provide an AI system. In particular, the technology disclosed proposes a parallel input, parallel output (PIPO) AI system based on the Transformer architecture. The Transformer model relies on a self-attention mechanism to compute a series of context-informed vector-space representations of elements in the input sequence and the output sequence, which are then used to predict distributions over subsequent elements as the model predicts the output sequence element-by-element. Not only is this mechanism straightforward to parallelize, but as each input's representation is also directly informed by all other inputs' representations, this results in an effectively global receptive field across the whole input sequence. This stands in contrast to, e.g., convolutional architectures which typically have a limited receptive field.

In one implementation, the disclosed AI system is a multilayer perceptron (MLP). In another implementation, the disclosed AI system is a feedforward neural network. In yet another implementation, the disclosed AI system is a fully connected neural network. In a further implementation, the disclosed AI system is a fully convolution neural network. In a yet further implementation, the disclosed AI system is a semantic segmentation neural network. In a yet another further implementation, the disclosed AI system is a generative adversarial network (GAN) (e.g., CycleGAN, StyleGAN, pixelRNN, text-2-image, DiscoGAN, IsGAN). In a yet another implementation, the disclosed AI system includes self-attention mechanisms like Transformer, Vision Transformer (ViT), Bidirectional Transformer (BERT), Detection Transformer (DETR), Deformable DETR, UP-DETR, DeiT, Swin, GPT, iGPT, GPT-2, GPT-3, various ChatGPT versions, various LLAMA versions, BERT, SpanBERT, ROBERTa, XLNet, ELECTRA, UniLM, BART, T5, ERNIE (THU), KnowBERT, DeiT-Ti, DeiT-S, DeiT-B, T2T-ViT-14, T2T-ViT-19, T2T-ViT-24, PVT-Small, PVT-Medium, PVT-Large, TNT-S, TNT-B, CPVT-S, CPVT-S-GAP, CPVT-B, Swin-T, Swin-S, Swin-B, Twins-SVT-S, Twins-SVT-B, Twins-SVT-L, Shuffle-T, Shuffle-S, Shuffle-B, XCiT-S12/16, CMT-S, CMT-B, VOLO-D1, VOLO-D2, VOLO-D3, VOLO-D4, MoCo v3, ACT, TSP, Max-DeepLab, VisTR, SETR, Hand-Transformer, HOT-Net, METRO, Image Transformer, Taming transformer, TransGAN, IPT, TTSR, STTN, Masked Transformer, CLIP, DALL-E, Cogview, UniT, ASH, TinyBert, FullyQT, ConvBert, FCOS, Faster R-CNN+FPN, DETR-DC5, TSP-FCOS, TSP-RCNN, ACT+MKDD (L=32), ACT+MKDD (L=16), SMCA, Efficient DETR, UP-DETR, UP-DETR, VITB/16-FRCNN, VIT-B/16-FRCNN, PVT-Small+RetinaNet, Swin-T+RetinaNet, Swin-T+ATSS, PVT-Small+DETR, TNT-S+DETR, YOLOS-Ti, YOLOS-S, and YOLOS-B.

In one implementation, the disclosed AI system is a convolution neural network (CNN) with a plurality of convolution layers. In another implementation, the disclosed AI system is a recurrent neural network (RNN) such as a long short-term memory network (LSTM), bi-directional LSTM (Bi-LSTM), or a gated recurrent unit (GRU). In yet another implementation, the disclosed AI system includes both a CNN and an RNN.

In yet other implementations, the disclosed AI system can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. The disclosed AI system can use one or more loss functions such as logistic regression/log loss, multi-class cross-entropy/softmax loss, binary cross-entropy loss, mean-squared error loss, L1 loss, L2 loss, smooth L1 loss, and Huber loss. The disclosed AI system can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). The disclosed AI system can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, and attention mechanisms.

The disclosed AI system can be a linear regression model, a logistic regression model, an Elastic Net model, a support vector machine (SVM), a random forest (RF), a decision tree, and a boosted decision tree (e.g., XGBoost), or some other tree-based logic (e.g., metric trees, kd-trees, R-trees, universal B-trees, X-trees, ball trees, locality sensitive hashes, and inverted indexes). The disclosed AI system can be an ensemble of multiple models, in some implementations.

In some implementations, the disclosed AI system can be trained using backpropagation-based gradient update techniques. Example gradient descent techniques that can be used for training the disclosed AI system include stochastic gradient descent, batch gradient descent, and mini-batch gradient descent. Some examples of gradient descent optimization algorithms that can be used to train the disclosed AI system are Momentum, Nesterov accelerated gradient, Adagrad, Adadelta, RMSprop, Adam, AdaMax, Nadam, and AMSGrad.

### Transformer Logic

Machine learning is the use and development of computer systems that can learn and adapt without following explicit instructions, by using algorithms and statistical models to analyze and draw inferences from patterns in data. Some of the state-of-the-art models use Transformers, a more powerful and faster model than neural networks alone. Transformers originate from the field of natural language processing (NLP), but can be used in computer vision and many other fields. Neural networks process input in series and weight relationships by distance in the series. Transformers can process input in parallel and do not necessarily weigh by distance. For example, in natural language processing, neural networks process a sentence from beginning to end with the weights of words close to each other being higher than those further apart. This leaves the end of the sentence very disconnected from the beginning causing an effect called the vanishing gradient problem. Transformers look at each word in parallel and determine weights for the relationships to each of the other words in the sentence. These relationships are called hidden states because they are later condensed for use into one vector called the context vector. Transformers can be used in addition to neural networks. This architecture is described here.

### Encoder-Decoder Architecture

FIG. 9 is a schematic representation of an encoder-decoder architecture. This architecture is often used for NLP and has two main building blocks. The first building block is the encoder that encodes an input into a fixed-size vector. In the system we describe here, the encoder is based on a recurrent neural network (RNN). At each time step, t, a hidden state of time step, t−1, is combined with the input value at time step t to compute the hidden state at timestep t. The hidden state at the last time step, encoded in a context vector, contains relationships encoded at all previous time steps. For NLP, each step corresponds to a word. Then the context vector contains information about the grammar and the sentence structure. The context vector can be considered a low-dimensional representation of the entire input space. For NLP, the input space is a sentence, and a training set consists of many sentences.

The context vector is then passed to the second building block, the decoder. For translation, the decoder has been trained on a second language. Conditioned on the input context vector, the decoder generates an output sequence. At each time step, t, the decoder is fed the hidden state of time step, t−1, and the output generated at time step, t−1. The first hidden state in the decoder is the context vector, generated by the encoder. The context vector is used by the decoder to perform the translation.

The whole model is optimized end-to-end by using backpropagation, a method of training a neural network in which the initial system output is compared to the desired output and the system is adjusted until the difference is minimized. In backpropagation, the encoder is trained to extract the right information from the input sequence, the decoder is trained to capture the grammar and vocabulary of the output language. This results in a fluent model that uses context and generalizes well. When training an encoder-decoder model, the real output sequence is used to train the model to prevent mistakes from stacking. When testing the model, the previously predicted output value is used to predict the next one.

When performing a translation task using the encoder-decoder architecture, all information about the input sequence is forced into one vector, the context vector. Information connecting the beginning of the sentence with the end is lost, the vanishing gradient problem. Also, different parts of the input sequence are important for different parts of the output sequence, information that cannot be learned using RNNs in an encoder-decoder architecture.

### Attention Mechanism

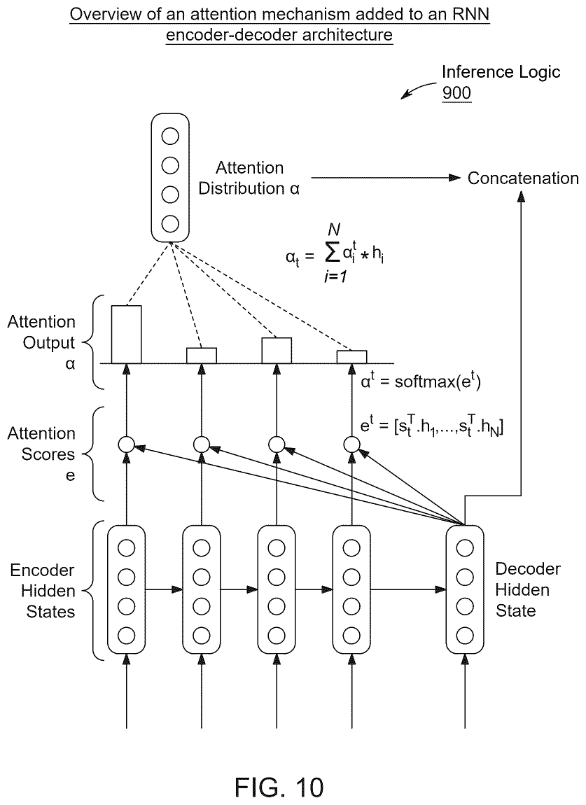

Attention mechanisms distinguish Transformers from other machine learning models. The attention mechanism provides a solution for the vanishing gradient problem. FIG. 10 shows an overview of an attention mechanism added onto an RNN encoder-decoder architecture. At every step, the decoder is given an attention score, e, for each encoder hidden state. In other words, the decoder is given weights for each relationship between words in a sentence. The decoder uses the attention score concatenated with the context vector during decoding. The output of the decoder at time step t is based on all encoder hidden states and the attention outputs. The attention output captures the relevant context for time step t from the original sentence. Thus, words at the end of a sentence may now have a strong relationship with words at the beginning of the sentence. In the sentence “The quick brown fox, upon arriving at the doghouse, jumped over the lazy dog,” fox and dog can be closely related despite being far apart in this complex sentence.

To weight encoder hidden states, a dot product between the decoder hidden state of the current time step, and all encoder hidden states, is calculated. This results in an attention score for every encoder hidden state. The attention scores are higher for those encoder hidden states that are similar to the decoder hidden state of the current time step. Higher values for the dot product indicate the vectors are pointing more closely in the same direction. The attention scores are converted to fractions that sum to one using the SoftMax function.

The SoftMax scores provide an attention distribution. The x-axis of the distribution is position in a sentence. The y-axis is attention weight. The scores show which encoder hidden states are most closely related. The SoftMax scores specify which encoder hidden states are the most relevant for the decoder hidden state of the current time step.

The elements of the attention distribution are used as weights to calculate a weighted sum over the different encoder hidden states. The outcome of the weighted sum is called the attention output. The attention output is used to predict the output, often in combination (concatenation) with the decoder hidden states. Thus, both information about the inputs, as well as the already generated outputs, can be used to predict the next outputs.

By making it possible to focus on specific parts of the input in every decoder step, the attention mechanism solves the vanishing gradient problem. By using attention, information flows more directly to the decoder. It does not pass through many hidden states. Interpreting the attention step can give insights into the data. Attention can be thought of as a soft alignment. The words in the input sequence with a high attention score align with the current target word. Attention describes long-range dependencies better than RNN alone. This enables analysis of longer, more complex sentences.

The attention mechanism can be generalized as: given a set of vector values and a vector query, attention is a technique to compute a weighted sum of the vector values, dependent on the vector query. The vector values are the encoder hidden states, and the vector query is the decoder hidden state at the current time step.

The weighted sum can be considered a selective summary of the information present in the vector values. The vector query determines on which of the vector values to focus. Thus, a fixed-size representation of the vector values can be created, in dependence upon the vector query.

The attention scores can be calculated by the dot product, or by weighing the different values (multiplicative attention).

### Embeddings

For most machine learning models, the input to the model needs to be numerical. The input to a translation model is a sentence, and words are not numerical. multiple methods exist for the conversion of words into numerical vectors. These numerical vectors are called the embeddings of the words. Embeddings can be used to convert any type of symbolic representation into a numerical one.

Embeddings can be created by using one-hot encoding. The one-hot vector representing the symbols has the same length as the total number of possible different symbols. Each position in the one-hot vector corresponds to a specific symbol. For example, when converting colors to a numerical vector, the length of the one-hot vector would be the total number of different colors present in the dataset. For each input, the location corresponding to the color of that value is one, whereas all the other locations are valued at zero. This works well for working with images. For NLP, this becomes problematic, because the number of words in a language is very large. This results in enormous models and the need for a lot of computational power. Furthermore, no specific information is captured with one-hot encoding. From the numerical representation, it is not clear that orange and red are more similar than orange and green. For this reason, other methods exist.

A second way of creating embeddings is by creating feature vectors. Every symbol has its specific vector representation, based on features. With colors, a vector of three elements could be used, where the elements represent the amount of yellow, red, and/or blue needed to create the color. Thus, all colors can be represented by using a vector of three elements. Also, similar colors have similar representation vectors.

For NLP, embeddings based on context, as opposed to words, are small and can be trained. The reasoning behind this concept is that words with similar meanings occur in similar contexts. Different methods take the context of words into account. Some methods, like GloVe, base their context embedding on co-occurrence statistics from corpora (large texts) such as Wikipedia. Words with similar co-occurrence statistics have similar word embeddings. Other methods use neural networks to train the embeddings. For example, they train their embeddings to predict the word based on the context (Common Bag of Words), and/or to predict the context based on the word (Skip-Gram). Training these contextual embeddings is time intensive. For this reason, pre-trained libraries exist. Other deep learning methods can be used to create embeddings. For example, the latent space of a variational autoencoder (VAE) can be used as the embedding of the input. Another method is to use 1D convolutions to create embeddings. This causes a sparse, high-dimensional input space to be converted to a denser, low-dimensional feature space.

### Self-Attention: Queries (Q), Keys (K), Values (V)

Transformer models are based on the principle of self-attention. Self-attention allows each element of the input sequence to look at all other elements in the input sequence and search for clues that can help it to create a more meaningful encoding. It is a way to look at which other sequence elements are relevant for the current element. The Transformer can grab context from both before and after the currently processed element.

When performing self-attention, three vectors need to be created for each element of the encoder input: the query vector (Q), the key vector (K), and the value vector (V). These vectors are created by performing matrix multiplications between the input embedding vectors using three unique weight matrices.

After this, self-attention scores are calculated. When calculating self-attention scores for a given element, the dot products between the query vector of this element and the key vectors of all other input elements are calculated. To make the model mathematically more stable, these self-attention scores are divided by the root of the size of the vectors. This has the effect of reducing the importance of the scalar thus emphasizing the importance of the direction of the vector. Just as before, these scores are normalized with a SoftMax layer. This attention distribution is then used to calculate a weighted sum of the value vectors, resulting in a vector z for every input element. In the attention principle explained above, the vector to calculate attention scores and to perform the weighted sum was the same, in self-attention two different vectors are created and used. As the self-attention needs to be calculated for all elements (thus a query for every element), one formula can be created to calculate a Z matrix. The rows of this Z matrix are the z vectors for every sequence input element, giving the matrix a size length sequence dimension QKV.

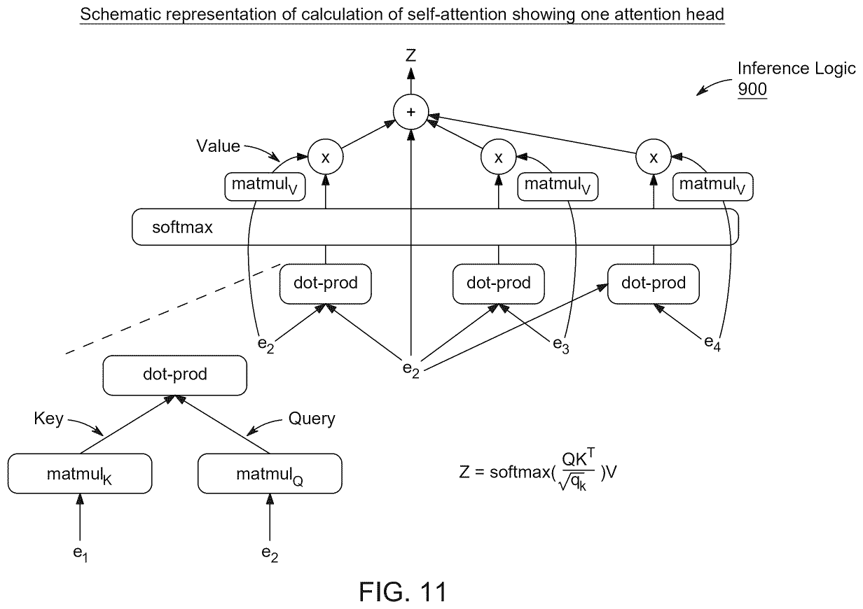

Multi-headed attention is executed in the Transformer. FIG. 11 is a schematic representation of the calculation of self-attention showing one attention head. For every attention head, different weight matrices are trained to calculate Q, K, and V. Every attention head outputs a matrix Z. Different attention heads can capture different types of information. The different Z matrices of the different attention heads are concatenated. This matrix can become large when multiple attention heads are used. To reduce dimensionality, an extra weight matrix W is trained to condense the different attention heads into a matrix with the same size as one Z matrix. This way, the amount of data given to the next step does not enlarge every time self-attention is performed.

When performing self-attention, information about the order of the different elements within the sequence is lost. To address this problem, positional encodings are added to the embedding vectors. Every position has its unique positional encoding vector. These vectors follow a specific pattern, which the Transformer model can learn to recognize. This way, the model can consider distances between the different elements.

As discussed above, in the core of self-attention are three objects: queries (Q), keys (K), and values (V). Each of these objects has an inner semantic meaning of their purpose. One can think of these as analogous to databases. We have a user-defined query of what the user wants to know. Then we have the relations in the database, i.e., the values which are the weights. More advanced database management systems create some apt representation of its relations to retrieve values more efficiently from the relations. This can be achieved by using indexes, which represent information about what is stored in the database. In the context of attention, indexes can be thought of as keys. So instead of running the query against values directly, the query is first executed on the indexes to retrieve where the relevant values or weights are stored. Lastly, these weights are run against the original values to retrieve data that is most relevant to the initial query.

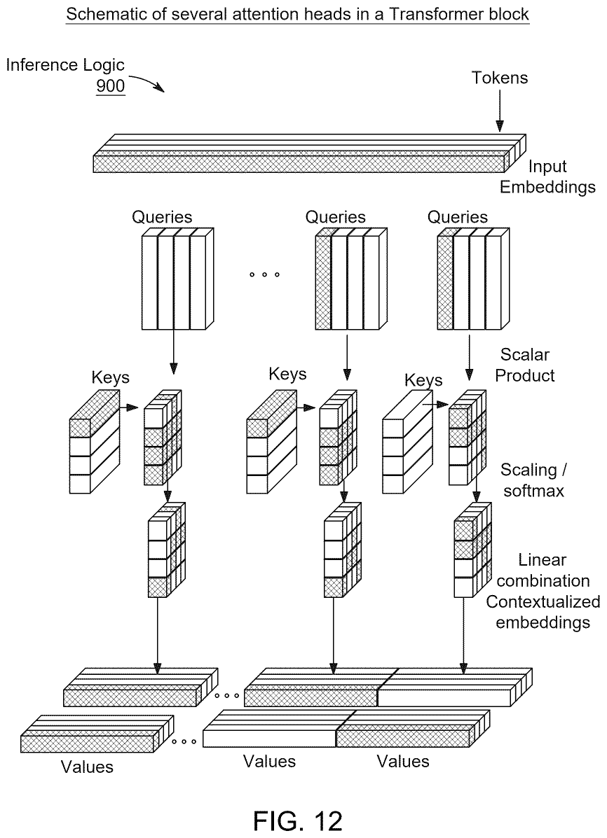

FIG. 12 depicts several attention heads in a Transformer block. We can see that the outputs of queries and keys dot products in different attention heads are differently colored. This depicts the capability of the multi-head attention to focus on different aspects of the input and aggregate the obtained information by multiplying the input with different attention weights.

Examples of attention calculation include scaled dot-product attention and additive attention. There are several reasons why scaled dot-product attention is used in the Transformers. Firstly, the scaled dot-product attention is relatively fast to compute, since its main parts are matrix operations that can be run on modern hardware accelerators. Secondly, it performs similarly well for smaller dimensions of the K matrix, dk, as the additive attention. For larger dk, the scaled dot-product attention performs a bit worse because dot products can cause the vanishing gradient problem. This is compensated via the scaling factor, which is defined as Vdk.

As discussed above, the attention function takes as input three objects: key, value, and query. In the context of Transformers, these objects are matrices of shapes (n, d), where n is the number of elements in the input sequence and dis the hidden representation of each element (also called the hidden vector). Attention is then computed as:

$$Attention ( Q , K , V ) = SoftMax ( \frac{{QK}^{T}}{\sqrt{dk}} ) V$$

where Q, K, V are computed as:

X·W_{Q},X·W_{K},X·W_{V}

X is the input matrix and W_{Q}, W_{K}, W_{V} are learned weights to project the input matrix into the representations. The dot products appearing in the attention function are exploited for their geometrical interpretation where higher values of their results mean that the inputs are more similar, i.e., pointing in the geometrical space in the same direction. Since the attention function now works with matrices, the dot product becomes matrix multiplication. The SoftMax function is used to normalize the attention weights into the value of 1 prior to being multiplied by the values matrix. The resulting matrix is used either as input into another layer of attention or becomes the output of the Transformer.

### Multi-Head Attention

Transformers become even more powerful when multi-head attention is used. Queries, keys, and values are computed the same way as above, though they are now projected into h different representations of smaller dimensions using a set of h learned weights. Each representation is passed into a different scaled dot-product attention block called a head. The head then computes its output using the same procedure as described above.

Formally, the multi-head attention is defined as:

MultiHeadAttention (Q, K, V)=[head₁, . . . , headₕ]_{W0} where headᵢ=Attention

$$( {QW}_{i}^{Q} , {KW}_{i}^{K} , {VW}_{i}^{V} )$$

The outputs of all heads are concatenated together and projected again using the learned weights matrix W₀ to match the dimensions expected by the next block of heads or the output of the Transformer. Using the multi-head attention instead of the simpler scaled dot-product attention enables Transformers to jointly attend to information from different representation subspaces at different positions.

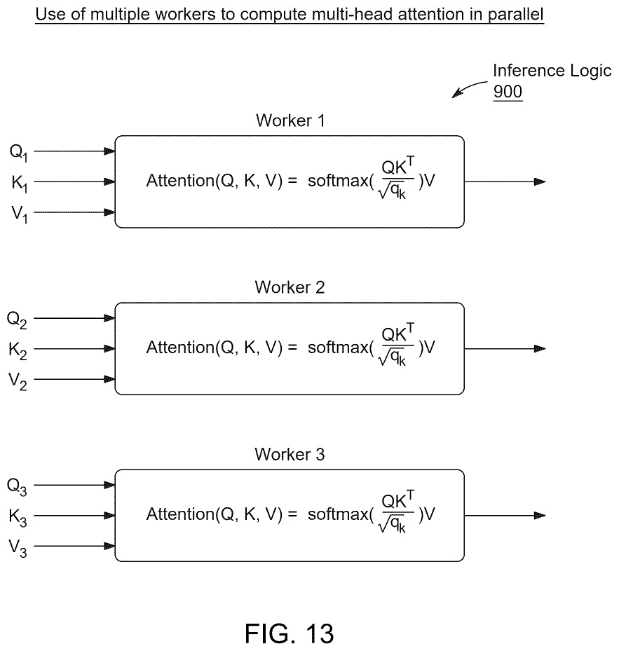

As shown in FIG. 13, one can use multiple workers to compute the multi-head attention in parallel, as the respective heads compute their outputs independently of one another. Parallel processing is one of the advantages of Transformers over RNNs.

Assuming the naive matrix multiplication algorithm which has a complexity of:

a·b·c

For matrices of shape (a, b) and (c, d), to obtain values Q, K, V, we need to compute the operations:

X·W_{Q},X·W_{K},X·WV

The matrix X is of shape (n, d) where n is the number of patches and dis the hidden vector dimension. The weights W_{Q}, W_{K}, W_{V} are all of shape (d, d). Omitting the constant factor 3, the resulting complexity is:

n·d²

We can proceed to the estimation of the complexity of the attention function itself, i.e., of

$$SoftMax ( \frac{{QK}^{T}}{\sqrt{dk}} ) V .$$

The matrices Q and K are both of shape (n, d). The transposition operation does not influence the asymptotic complexity of computing the dot product of matrices of shapes (n, d)·(d, n), therefore its complexity is:

n²·d

Scaling by a constant factor of \sqrt{dk}, where dk is the dimension of the keys vector, as well as applying the SoftMax function, both have the complexity of a·b for a matrix of shape (a, b), hence they do not influence the asymptotic complexity. Lastly the dot product SoftMax

$$( \frac{{QK}^{T}}{\sqrt{dk}} ) \cdot V$$

is between matrices of shapes (n, n) and (n, d) and so its complexity is:

n²·d

The final asymptotic complexity of scaled dot-product attention is obtained by summing the complexities of computing Q, K, V, and of the following attention function:

n·d²+n²·d.

The asymptotic complexity of multi-head attention is the same since the original input matrix X is projected into h matrices of shapes

$$( n , \frac{d}{h} ) ,$$

where h is the number of heads. From the point of view of asymptotic complexity, h is constant, therefore we would arrive at the same estimate of asymptotic complexity using a similar approach as for the scaled dot-product attention.

Transformer models often have the encoder-decoder architecture, although this is not necessarily the case. The encoder is built out of different encoder layers which are all constructed in the same way. The positional encodings are added to the embedding vectors. Afterward, self-attention is performed.

### Encoder Block of Transformer

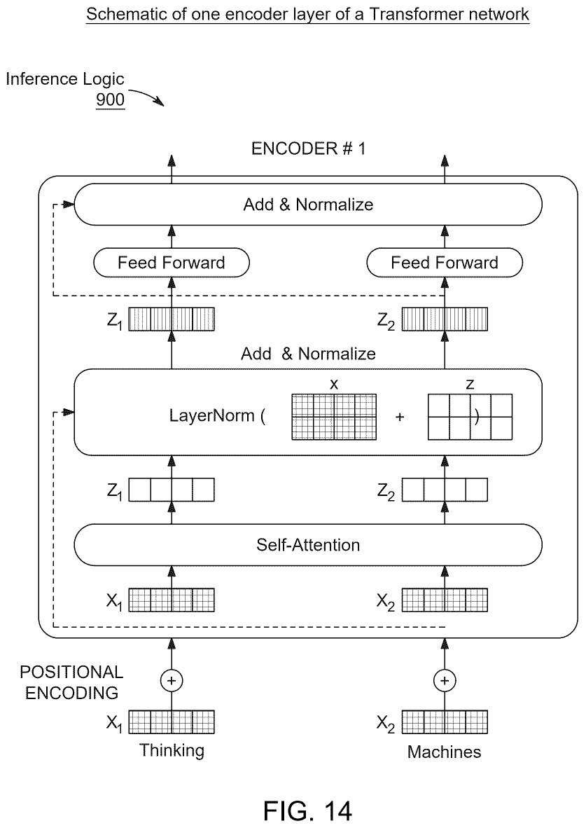

FIG. 14 portrays one encoder layer of a Transformer network. Every self-attention layer is surrounded by a residual connection, summing up the output and input of the self-attention. This sum is normalized, and the normalized vectors are fed to a feed-forward layer. Every z vector is fed separately to this feed-forward layer. The feed-forward layer is wrapped in a residual connection and the outcome is normalized too. Often, numerous encoder layers are piled to form the encoder. The output of the encoder is a fixed-size vector for every element of the input sequence.

Just like the encoder, the decoder is built from different decoder layers. In the decoder, a modified version of self-attention takes place. The query vector is compared to the keys of previous output sequence elements. The elements further in the sequence are not known yet, as they still must be predicted. No information about these output elements may be used.

### Encoder-Decoder Blocks of Transformer

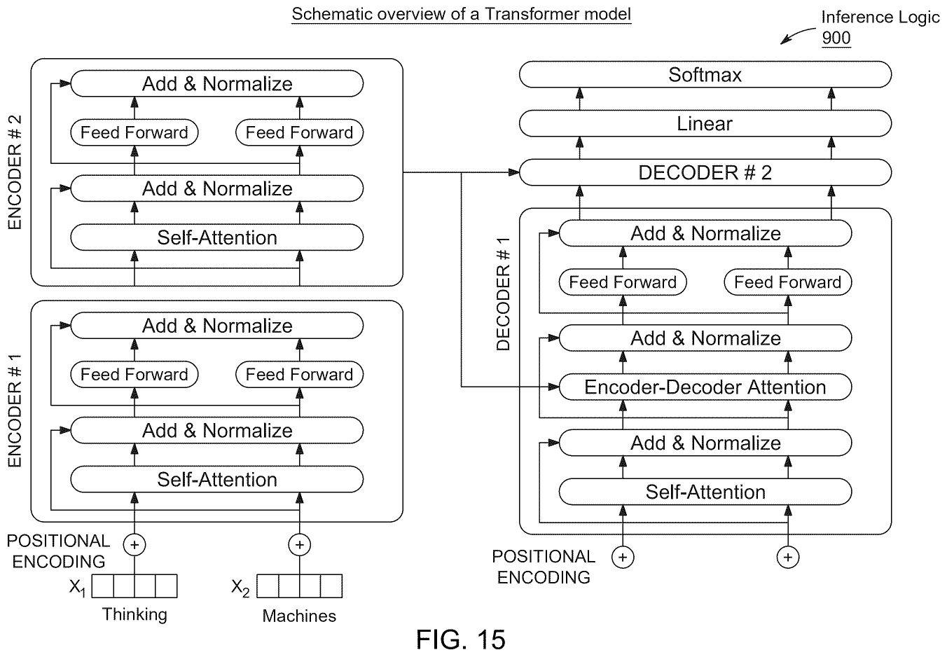

FIG. 15 shows a schematic overview of a Transformer model. Next to a self-attention layer, a layer of encoder-decoder attention is present in the decoder, in which the decoder can examine the last Z vectors of the encoder, providing fluent information transmission. The ultimate decoder layer is a feed-forward layer. All layers are packed in a residual connection. This allows the decoder to examine all previously predicted outputs and all encoded input vectors to predict the next output. Thus, information from the encoder is provided to the decoder, which could improve the predictive capacity. The output vectors of the last decoder layer need to be processed to form the output of the entire system. This is done by a combination of a feed-forward layer and a SoftMax function. The output corresponding to the highest probability is the predicted output value for a subject time step.

For some tasks other than translation, an encoder is needed. This is true for both document classification and name entity recognition. In these cases, the encoded input vectors are the input of the feed-forward layer and the SoftMax layer. Transformer models have been extensively applied in different NLP fields, such as translation, document summarization, speech recognition, and named entity recognition. These models have applications in the field of biology as well for predicting protein structure and function and labeling DNA sequences.

### Vision Transformer

There are extensive applications of transformers in vision including popular recognition tasks (e.g., image classification, object detection, action recognition, and segmentation), generative modeling, multi-modal tasks (e.g., visual-question answering, visual reasoning, and visual grounding), video processing (e.g., activity recognition, video forecasting), low-level vision (e.g., image super-resolution, image enhancement, and colorization) and 3D analysis (e.g., point cloud classification and segmentation).

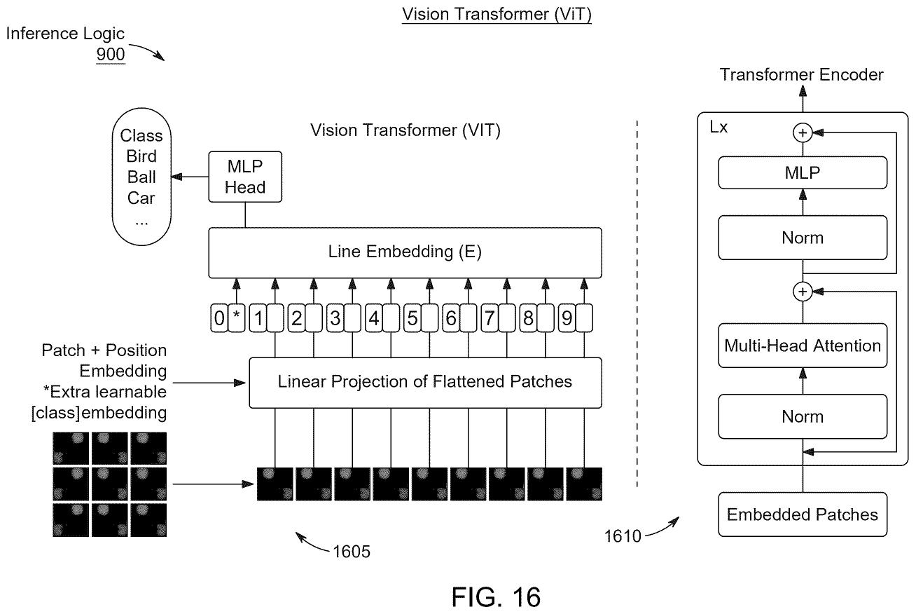

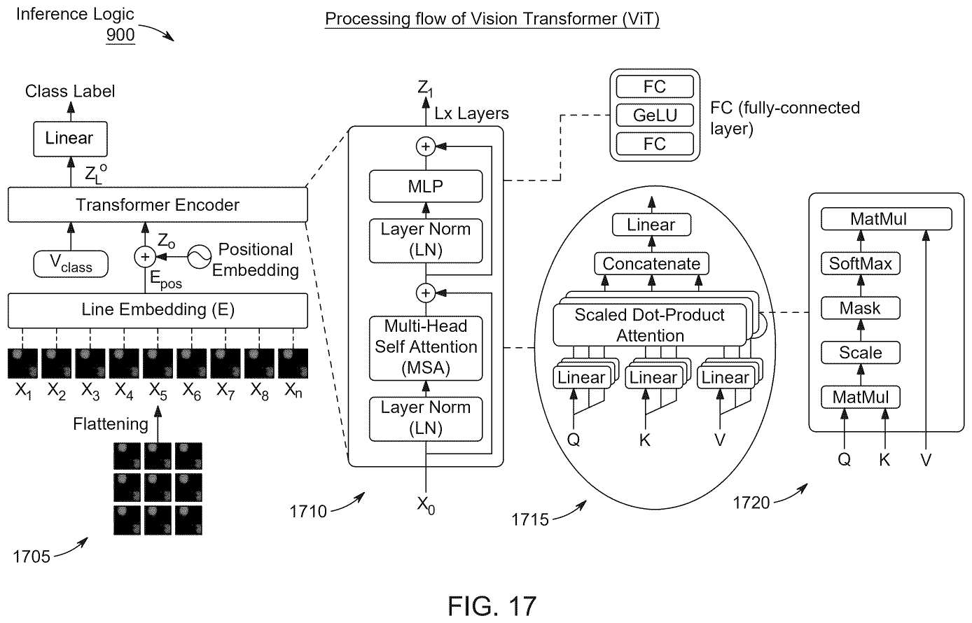

Transformers were originally developed for NLP and worked with sequences of words. In image classification, we often have a single input image in which the pixels are in a sequence. To reduce the computation required, Vision Transformers (ViTs) cut the input image into a set of fixed-sized patches of pixels. The patches are often 16×16 pixels. They are treated much like words in NLP Transformers. ViTs are depicted in FIG. 16 (1605, 1610), and FIG. 17 (1705, 1710, 1715, 1720). Unfortunately, important positional information is lost because image sets are position-invariant. This problem is solved by adding a learned positional encoding into the image patches.

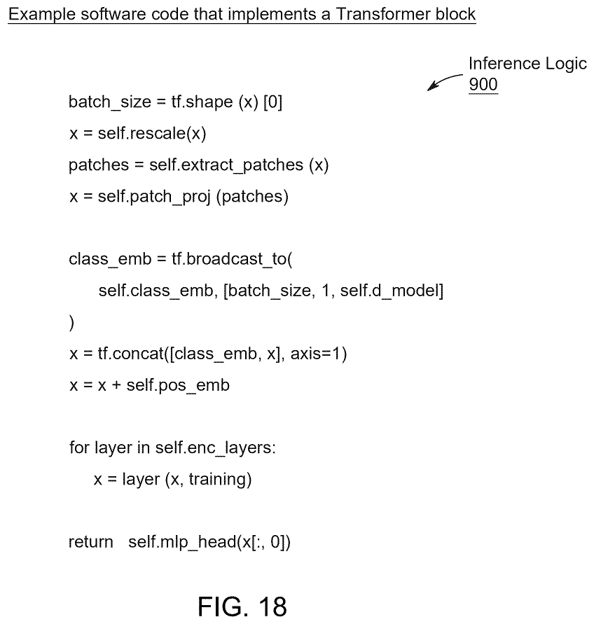

The computations of the ViT architecture can be summarized as follows. The first layer of a ViT extracts a fixed number of patches from an input image (1605). The patches are then projected to linear embeddings. A special class token vector is added to the sequence of embedding vectors to include all representative information of all tokens through the multi-layer encoding procedure. The class vector is unique to each image. Vectors containing positional information are combined with the embeddings and the class token. The sequence of embedding vectors is passed into the Transformer blocks. The class token vector is extracted from the output of the last Transformer block and is passed into a multilayer perceptron (MLP) head whose output is the final classification. The perceptron takes the normalized input and places the output in categories. It classifies the images. This procedure directly translates into the Python Keras code shown in FIG. 18.

When the input image is split into patches, a fixed patch size is specified before instantiating a ViT. Given the quadratic complexity of attention, patch size has a large effect on the length of training and inference time. A single Transformer block comprises several layers. The first layer implements Layer Normalization, followed by the multi-head attention that is responsible for the performance of ViTs. In the depiction of a Transformer block 2510, we can see two arrows. These are residual skip connections. Including skip connection data can simplify the output and improve the results. The output of the multi-head attention is followed again by Layer Normalization. And finally, the output layer is an MLP (Multi-Layer Perceptron) with the GELU (Gaussian Error Linear Unit) activation function.

ViTs can be pretrained and fine-tuned. Pretraining is generally done on a large dataset. Fine-tuning is done on a domain specific dataset.

Domain-specific architectures, like convolutional neural networks (CNNs) or long short-term memory networks (LSTMs), have been derived from the usual architecture of MLPs and suffer from so-called inductive biases that predispose the networks towards a certain output. ViTs stepped in the opposite direction of CNNs and LSTMs and became more general architectures by eliminating inductive biases. A ViT can be seen as a generalization of MLPs because MLPs, after being trained, do not change their weights for different inputs. On the other hand, ViTs compute their attention weights at runtime based on the particular input.

### Computer System

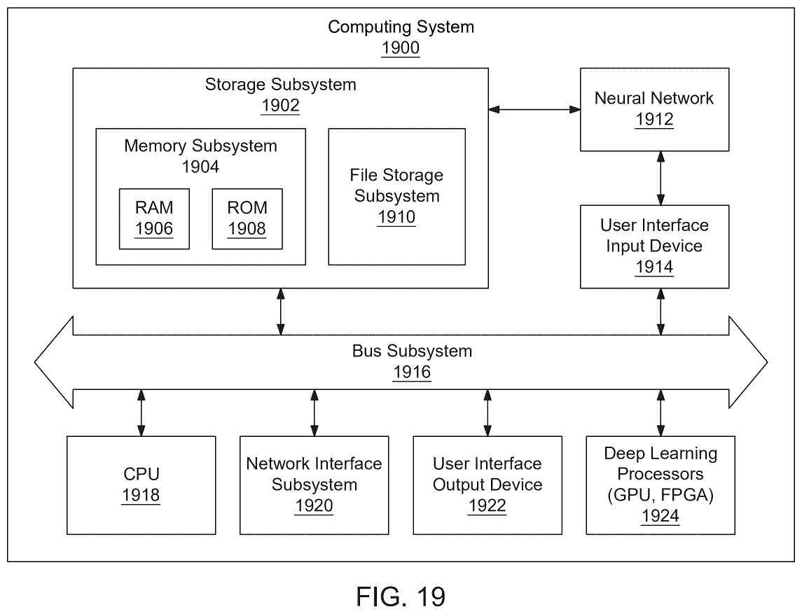

FIG. 19 shows an example computer system 1900 that can be used to implement the technology disclosed. The computer system 1900 is a representation of the system, as described in FIG. 4. The computer system 1900 includes at least one central processing unit (CPU) 1918 that communicates with a number of peripheral devices via bus subsystem 1916. These peripheral devices can include a storage subsystem 1902 including, for example, memory devices and a file storage subsystem 1910, user interface input devices 1914, user interface output devices 1922, and a network interface subsystem 1920. The input and output devices allow user interaction with computer system 1900. Network interface subsystem 1920 provides an interface to outside networks, including an interface to corresponding interface devices in other computer systems.

In one implementation, a neural network 1912 is communicably linked to the storage subsystem 1902 and the user interface input devices 1914.

User interface input devices 1914 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 1900.

User interface output devices 1922 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 1900 to the user or to another machine or computer system.

Storage subsystem 1902 stores programming and data constructs that provide the functionality of some or all of the modules and methods described herein. These software modules are generally executed by deep learning processors 1924.

Deep learning processors 1924 can be graphics processing units (GPUs), field-programmable gate arrays (FPGAs), application-specific integrated circuits (ASICs), and/or coarse-grained reconfigurable architectures (CGRAs). Processors 1924 can be hosted by a deep learning cloud platform such as Google Cloud Platform™, Xilinx™, and Cirrascale™ Examples of processors 1924 include Google's Tensor Processing Unit (TPU)™, rackmount solutions like GX4 Rackmount Series™, GX6 Rackmount Series™, NVIDIA DGX-1™, Microsoft's Stratix V FPGA™, Graphcore's Intelligent Processor Unit (IPU)™, Qualcomm's Zeroth Platform™ with Snapdragon Processors™, NVIDIA's Volta™, NVIDIA's DRIVE PX™, NVIDIA's JETSON TX1/TX2 MODULE™, Intel's Nirvana™, Movidius VPU™, Fujitsu DPI™, ARM's DynamicIQ™, IBM TrueNorth™, Lambda GPU Server with Testa V100s™, and others.

Memory subsystem 1904 used in the storage subsystem 1902 can include a number of memories including a main random-access memory (RAM) 1906 for storage of instructions and data during program execution and a read only memory (ROM) 1908 in which fixed instructions are stored. A file storage subsystem 1910 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 1910 in the storage subsystem 1902, or in other machines accessible by the processor.

Bus subsystem 1916 provides a mechanism for letting the various components and subsystems of computer system 1900 communicate with each other as intended. Although bus subsystem 1916 is shown schematically as a single bus, alternative implementations of the bus subsystem can use multiple buses.

Computer system 1900 itself can be of varying types including a personal computer, a portable computer, a workstation, a computer terminal, a network computer, a television, a mainframe, a server farm, a widely distributed set of loosely networked computers, or any other data processing system or user device. Due to the ever-changing nature of computers and networks, the description of computer system 1900 depicted in FIG. 19 is intended only as a specific example for the purpose of illustrating the preferred implementations of the present technology disclosed. Many other configurations of computer system 1900 are possible having more or fewer components than the computer system depicted in FIG. 19.

In various implementations, a learning system is provided. In some implementations, a feature vector is provided to a learning system. Based on the input features, the learning system generates one or more outputs. In some implementations, the output of the learning system is a feature vector. In some implementations, the learning system comprises an SVM. In other implementations, the learning system comprises an artificial neural network. In some implementations, the learning system is pre-trained using training data. In some implementations training data is retrospective data. In some implementations, the retrospective data is stored in a data store. In some implementations, the learning system may be additionally trained through manual curation of previously generated outputs.

In some implementations, an object detection pipeline is a trained classifier. In some implementations, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer system/server may be described in the general context of computer system-executable instructions, such as program modules, being executed by a computer system. Generally, program modules may include routines, programs, objects, components, logic, data structures, and so on that perform particular tasks or implement particular abstract data types. Computer system/server may be practiced in distributed computing environments where tasks are performed by remote processing devices that are linked through a communications network. In a distributed computing environment, program modules may be located in both local and remote computer system storage media including memory storage devices.

As shown in FIG. 19, computer system/server in computing node is shown in the form of a general-purpose computing device. The components of computer system/server may include, but are not limited to, one or more processors or processing units, a system memory, and a bus that couples various system components including system memory to processor.

The bus represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server typically includes a variety of computer system readable media. Such media may be any available media that is accessible by computer system/server, and it includes both volatile and non-volatile media, removable and non-removable media.

System memory can include computer system readable media in the form of volatile memory, such as random access memory (RAM) and/or cache memory. Algorithm Computer system/server may further include other removable/non-removable, volatile/non-volatile computer system storage media. By way of example only, storage system can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to the bus by one or more data media interfaces. As will be further depicted and described below, memory may include at least one program product having a set (e.g., at least one) of program modules that are configured to carry out the functions of embodiments of the disclosure.

Program/utility, having a set (at least one) of program modules, may be stored in memory by way of example, and not limitation, as well as an operating system, one or more application programs, other program modules, and program data. Each of the operating system, one or more application programs, other program modules, and program data or some combination thereof, may include an implementation of a networking environment. Program modules generally carry out the functions and/or methodologies of embodiments as described herein.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some implementations, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

One or more implementations and clauses of the technology disclosed, or elements thereof can be implemented in the form of a computer product, including a non-transitory computer readable storage medium with computer usable program code for performing the method steps indicated. Furthermore, one or more implementations and clauses of the technology disclosed, or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

### CLAUSES

1. A method of operating a medication dispensing apparatus, the method comprising:
   obtaining, by one or more processors, a plurality of records associated with a user, the plurality of records comprising at least a record identifying a medication and a record indicating an assignment of the medication to a storage compartment of the medication dispensing apparatus;
   deriving, by the one or more processors, an identifier from the plurality of records;
   generating, by the one or more processors, a control instruction addressing the storage compartment, the control instruction being conditioned on a correspondence between the assignment and the storage compartment;
   transmitting, by the one or more processors, the control instruction to the medication dispensing apparatus to cause a state change of the storage compartment; and
   recording an event record linked to the identifier and indicating an outcome of the state change.
2. The method of clause 1, further comprising receiving sensor data from the medication dispensing apparatus and computing a consumed-units quantity for the medication from the sensor data.
3. The method of clause 1, wherein at least one of the plurality of records is obtained by applying a trained inference model to image data.
4. The method of clause 1, further comprising generating a verification result based on a comparison of a consumed-units quantity for the medication to an expected-units quantity, wherein a subsequent control instruction is conditioned on the verification result.
5. The method of clause 1, further comprising receiving biometric signals associated with the user, computing a health-context value from the biometric signals, and conditioning a subsequent control instruction on the health-context value.
6. The method of clause 1, wherein the event record is encrypted at rest on the non-transitory storage medium and is synchronized at a predetermined interval with a cloud-based audit store through a communications interface.
7. The method of clause 1, further comprising defining a sequence of temporal states for a scheduled dispensing event of the medication and transmitting a notification to a recipient distinct from the user upon entry into a defined temporal state of the sequence of temporal states.
8. A system comprising:
   one or more processors;
   one or more storage media coupled to the one or more processors and storing instructions; and
   a communications interface configured to communicate with a medication dispensing apparatus,
   the instructions, when executed by the one or more processors, causing the system to:
   obtain a plurality of records associated with a user, the plurality of records comprising at least a record identifying a medication and a record indicating an assignment of the medication to a storage compartment of the medication dispensing apparatus,
   derive an identifier from the plurality of records,
   generate a control instruction addressing the storage compartment, the control instruction being conditioned on a correspondence between the assignment and the storage compartment,
   transmit, through the communications interface, the control instruction to the medication dispensing apparatus to cause a state change of the storage compartment, and
   record an event record linked to the identifier and indicating an outcome of the state change.
9. The system of clause 8, further comprising the medication dispensing apparatus.
10. The system of clause 8, further comprising a wireless interface coupled to the one or more processors and configured to receive biometric signals associated with the user.
11. The system of clause 8, wherein the instructions further cause the system to receive sensor data from the medication dispensing apparatus and to compute a consumed-units quantity for the medication from the sensor data.
12. The system of clause 8, wherein the instructions further cause the system to encrypt the event record at rest on the one or more storage media and to synchronize the event record at a predetermined interval with a cloud-based audit store through the communications interface.
13. The system of clause 8, wherein the instructions further cause the system to compute a health-context value from biometric signals associated with the user and to condition a subsequent control instruction on the health-context value.
14. The system of clause 8, wherein the instructions further cause the system to apply a trained inference model to image data to obtain at least one of the plurality of records.
15. One or more non-transitory computer-readable storage media storing instructions that, when executed by one or more processors, cause the one or more processors to perform operations comprising:
   obtaining a plurality of records associated with a user, the plurality of records comprising at least a record identifying a medication and a record indicating an assignment of the medication to a storage compartment of a medication dispensing apparatus;
   deriving an identifier from the plurality of records;
   generating a control instruction addressing the storage compartment, the control instruction being conditioned on a correspondence between the assignment and the storage compartment;
   transmitting the control instruction to the medication dispensing apparatus to cause a state change of the storage compartment; and
   recording an event record linked to the identifier and indicating an outcome of the state change.
16. The one or more non-transitory computer-readable storage media of clause 15, wherein the operations further comprise receiving sensor data from the medication dispensing apparatus and computing a consumed-units quantity for the medication from the sensor data.
17. The one or more non-transitory computer-readable storage media of clause 15, wherein the operations further comprise applying a trained inference model to image data to obtain at least one of the plurality of records.
18. The one or more non-transitory computer-readable storage media of clause 15, wherein the operations further comprise computing a health-context value from biometric signals associated with the user and conditioning a subsequent control instruction on the health-context value.
19. The one or more non-transitory computer-readable storage media of clause 15, wherein the event record is encrypted at rest on the one or more storage media and is synchronized at a predetermined interval with a cloud-based audit store.
20. The one or more non-transitory computer-readable storage media of clause 15, wherein the operations further comprise defining a sequence of temporal states for a scheduled dispensing event of the medication and transmitting a notification to a recipient distinct from the user upon entry into a defined temporal state of the sequence of temporal states.
21. A method of operating a medication dispensing apparatus having a plurality of compartments, the method comprising:
   obtaining, by one or more processors, a user profile record, a device identifier of a peripheral data source paired with a user, a structured prescription record for a medication associated with a medication container, and a placement record indicating an assignment of the medication container to a designated compartment of the plurality of compartments;
   deriving, by the one or more processors, a composite identifier from the user profile record, the device identifier, the structured prescription record, and the placement record;
   generating, by the one or more processors, a control instruction addressing the designated compartment for a dispensing operation of the medication, the control instruction being conditioned on a correspondence between the placement record and the designated compartment addressed by the control instruction;
   transmitting, by the one or more processors through a communications interface to the medication dispensing apparatus, the control instruction to cause a physical state change of the designated compartment; and
   recording, on a non-transitory storage medium, an event record linked to the composite identifier and indicating an outcome of the physical state change received from the medication dispensing apparatus.
22. The method of clause 21, wherein the structured prescription record is obtained by applying a trained inference model to image data of a prescription label affixed to the medication container to extract a plurality of prescription fields, and is validated against a pharmacological reference data store comprising drug-interaction data or contraindication data.
23. The method of clause 22, wherein the image data comprises a multi-angle image sequence of the prescription label captured during a continuous rotation of the medication container relative to an image sensor.
24. The method of clause 21, further comprising:
   receiving, from a weight sensor of the medication dispensing apparatus, a baseline container weight measurement and a current container weight measurement of the medication container;
   computing a consumed-units quantity from a weight differential between the baseline container weight measurement and the current container weight measurement and a per-unit-mass value associated with the medication;
   determining an initial-units quantity by selecting, in a predetermined precedence order, from (i) an initial-units field of the structured prescription record, (ii) a quantity derived from a difference between the baseline container weight measurement and a reference empty-container weight retrieved from a reference data store of empty-container weights using a container category of the medication container, and (iii) a default value; and
   computing a remaining-units quantity from a difference between the initial-units quantity and the consumed-units quantity.
25. The method of clause 24, further comprising generating a verification result from a comparison of the consumed-units quantity to an expected-units quantity derived from the structured prescription record, wherein a subsequent control instruction directed to the designated compartment modifies a subsequent dispensing operation of the designated compartment when the verification result indicates a discrepancy exceeding a configurable tolerance value stored on the non-transitory storage medium.
26. The method of clause 21, further comprising:
   receiving, from the peripheral data source, a plurality of biometric signals for the user;
   for each biometric signal of the plurality of biometric signals, producing a contextual weight value by combining a contextual factor vector with a set of sensitivity coefficients, the contextual factor vector comprising a regimen-status value, and producing a normalized biometric value by applying a bounded-interval normalization;
   aggregating, across the plurality of biometric signals, products of the contextual weight values and the normalized biometric values to produce a wellness score; and
   updating the regimen-status value from the event record such that a subsequent wellness score reflects the event record, wherein a subsequent control instruction directed to the designated compartment is based at least in part on the wellness score.
27. The method of clause 21, further comprising defining, for a scheduled dispensing event of the medication, a sequence of temporal states comprising at least a first temporal state, a second temporal state, and a third temporal state ordered by increasing elapsed time from a scheduled time, each temporal state being associated with a distinct output pattern, and upon entry into the third temporal state, transmitting a notification to a notification recipient of a recipient hierarchy comprising at least one recipient distinct from the user.
28. A system comprising:
   one or more processors;
   one or more non-transitory storage media coupled to the one or more processors and storing instructions; and
   a communications interface coupled to the one or more processors and configured to exchange data with a medication dispensing apparatus having a plurality of compartments,
   the instructions, when executed by the one or more processors, causing the system to:
   obtain a user profile record, a device identifier of a peripheral data source paired with a user, a structured prescription record for a medication, and a placement record indicating an assignment of a medication container to a designated compartment of the plurality of compartments,
   derive a composite identifier from the user profile record, the device identifier, the structured prescription record, and the placement record,
   generate a control instruction addressing the designated compartment for a dispensing operation of the medication, the control instruction being conditioned on a correspondence between the placement record and the designated compartment addressed by the control instruction,
   transmit, through the communications interface, the control instruction to cause a physical state change of the designated compartment, and
   record, on the one or more non-transitory storage media, an event record linked to the composite identifier and indicating an outcome of the physical state change received from the medication dispensing apparatus.
29. The system of clause 28, further comprising the medication dispensing apparatus, the medication dispensing apparatus comprising a rotatable storage member carrying the plurality of compartments, a movable access member configured to expose the designated compartment at a dispensing position, at least one actuator coupled to the movable access member and responsive to the control instruction, and a weight sensor configured to measure a weight of the medication container.
30. The system of clause 29, wherein the medication dispensing apparatus further comprises a configuration-encoding interface configured to communicate an installed compartment count of the plurality of compartments through the communications interface, and the instructions further cause the system to set an indexing of the plurality of compartments based on the installed compartment count.
31. The system of clause 28, wherein the instructions further cause the system to obtain the structured prescription record by applying a trained inference model to image data of a prescription label to extract a plurality of prescription fields, the trained inference model being trained on a corpus comprising printed prescription label images and handwritten prescription label images.
32. The system of clause 28, further comprising a wireless interface coupled to the one or more processors and configured to receive a plurality of biometric signals from the peripheral data source, wherein the instructions further cause the system to compute a wellness score from the plurality of biometric signals using a contextual factor vector comprising a regimen-status value updated from the event record, and to generate a subsequent control instruction directed to the designated compartment based at least in part on the wellness score.
33. The system of clause 28, wherein the instructions further cause the system to localize the structured prescription record into a region-appropriate format based on a jurisdictional compliance rule set stored on the one or more non-transitory storage media prior to recording the event record.
34 One or more non-transitory computer-readable storage media storing instructions that, when executed by one or more processors, cause the one or more processors to perform operations comprising:
   obtaining a user profile record, a device identifier of a peripheral data source paired with a user, a structured prescription record for a medication, and a placement record indicating an assignment of a medication container to a designated compartment of a plurality of compartments of a medication dispensing apparatus;
   deriving a composite identifier from the user profile record, the device identifier, the structured prescription record, and the placement record;
   generating a control instruction addressing the designated compartment for a dispensing operation of the medication, the control instruction being conditioned on a correspondence between the placement record and the designated compartment addressed by the control instruction;
   transmitting the control instruction to the medication dispensing apparatus to cause a physical state change of the designated compartment; and
   recording an event record linked to the composite identifier and indicating an outcome of the physical state change received from the medication dispensing apparatus.
35. The one or more non-transitory computer-readable storage media of clause 34, wherein the operations further comprise:
   receiving, from a weight sensor of the medication dispensing apparatus, a baseline container weight measurement and a current container weight measurement of the medication container;
   computing a consumed-units quantity from a weight differential between the baseline container weight measurement and the current container weight measurement and a per-unit-mass value associated with the medication;
   determining an initial-units quantity by selecting, in a predetermined precedence order, from (i) an initial-units field of the structured prescription record, (ii) a quantity derived from a difference between the baseline container weight measurement and a reference empty-container weight retrieved from a reference data store of empty-container weights using a container category of the medication container, and (iii) a default value; and
   generating a verification result from a comparison of the consumed-units quantity to an expected-units quantity derived from the structured prescription record, wherein a subsequent control instruction directed to the designated compartment modifies a subsequent dispensing operation of the designated compartment when the verification result indicates a discrepancy exceeding a configurable tolerance value.
36. The one or more non-transitory computer-readable storage media of clause 34, wherein the operations further comprise computing a wellness score from a plurality of biometric signals received from the peripheral data source, the wellness score being computed using a contextual factor vector comprising a regimen-status value updated from the event record, and generating a subsequent control instruction directed to the designated compartment based at least in part on the wellness score.
37. The one or more non-transitory computer-readable storage media of clause 34, wherein the operations further comprise encrypting the event record at rest on the one or more non-transitory computer-readable storage media in conformance with applicable data privacy requirements, and synchronizing the event record at a predetermined interval with a cloud-based audit store.
38. The one or more non-transitory computer-readable storage media of clause 34, wherein the operations further comprise:
   defining, for a scheduled dispensing event of the medication, a sequence of temporal states ordered by increasing elapsed time from a scheduled time, the sequence comprising at least a first temporal state, a second temporal state, and a third temporal state, each temporal state being associated with a distinct output pattern;
   driving an output subsystem of the medication dispensing apparatus through the output patterns according to a current temporal state;
   in response to receiving a snooze response, resetting the sequence to the first temporal state a number of times bounded by a predetermined maximum; and
   upon entry into the third temporal state, transmitting a notification to a notification recipient of a recipient hierarchy comprising at least one recipient distinct from the user.
39. The one or more non-transitory computer-readable storage media of clause 34, wherein the operations further comprise obtaining the structured prescription record by applying a trained inference model to a multi-angle image sequence of a prescription label captured during a continuous rotation of the medication container relative to an image sensor.
40. The one or more non-transitory computer-readable storage media of clause 35, wherein modifying the subsequent dispensing operation of the designated compartment comprises at least one of halting the subsequent dispensing operation, delaying the subsequent dispensing operation, or requiring an additional authorization for the subsequent dispensing operation.
41. A method of operating a medication dispensing apparatus, the medication dispensing apparatus comprising a rotatable storage member carrying a plurality of compartments, a movable access member configured to expose a designated compartment of the plurality of compartments at a dispensing position, at least one actuator coupled to the movable access member, a weight sensor configured to measure a weight of a medication container containing a medication and received in the designated compartment, an image sensor, a configuration-encoding interface, and a communications interface, the method comprising:
   receiving, by one or more processors from the image sensor, a multi-angle image sequence of a prescription label affixed to the medication container, the multi-angle image sequence captured during a continuous rotation of the medication container relative to the image sensor in a counterclockwise direction;
   applying, by the one or more processors, a trained inference model to the multi-angle image sequence to extract a plurality of prescription fields including a drug name, a dosage, a frequency, a duration, a prescribing physician, and an initial-units field, the trained inference model being trained on a corpus comprising printed prescription label images and handwritten prescription label images;
   validating, by the one or more processors, the plurality of prescription fields against a pharmacological reference data store comprising drug-interaction data and contraindication data to produce a structured prescription record;
   obtaining, by the one or more processors, a user profile record of a user, a device identifier of a peripheral data source paired with the user, and a placement record indicating an assignment of the medication container to the designated compartment;
   deriving, by the one or more processors, a composite identifier from the user profile record, the device identifier, the structured prescription record, and the placement record;
   receiving, by the one or more processors through the communications interface from the configuration-encoding interface, an installed compartment count of the plurality of compartments at power-on of the medication dispensing apparatus, and setting an indexing of the plurality of compartments based on the installed compartment count;
   generating, by the one or more processors at a scheduled dispensing time of the medication, a control instruction addressing the designated compartment for a dispensing operation of the medication, the control instruction being conditioned on a correspondence between the placement record and the designated compartment addressed by the control instruction;
   transmitting, by the one or more processors through the communications interface to the medication dispensing apparatus, the control instruction to cause the at least one actuator to drive the movable access member to expose the designated compartment at the dispensing position;
   receiving, by the one or more processors through the communications interface from the weight sensor, a baseline container weight measurement of the medication container before the dispensing operation and a current container weight measurement of the medication container after the dispensing operation;
   computing, by the one or more processors, a consumed-units quantity from a weight differential between the baseline container weight measurement and the current container weight measurement and a per-unit-mass value associated with the medication;
   determining, by the one or more processors, an initial-units quantity by selecting, in a predetermined precedence order, from (i) the initial-units field of the structured prescription record when the initial-units field is populated, (ii) a quantity derived from a difference between the baseline container weight measurement and a reference empty-container weight retrieved from a reference data store of empty-container weights using a container category of the medication container when the initial-units field is not populated and the reference data store contains an entry for the container category, and (iii) a default value of one hundred units when neither prior source applies;
   computing, by the one or more processors, a remaining-units quantity from a difference between the initial-units quantity and the consumed-units quantity;
   generating, by the one or more processors, a verification result from a comparison of the consumed-units quantity to an expected-units quantity derived from the structured prescription record, and when the verification result indicates a discrepancy exceeding a configurable tolerance value, generating a subsequent control instruction modifying a subsequent dispensing operation of the designated compartment;
   receiving, by the one or more processors from the peripheral data source, a plurality of biometric signals comprising at least a heart-rate signal, an activity signal, and a sleep signal;
   for each biometric signal of the plurality of biometric signals, producing a contextual weight value by combining a contextual factor vector with a set of sensitivity coefficients, the contextual factor vector comprising a regimen-status value, an activity-level value, a symptom-severity value, a time-of-day value, a historical-deviation value, an environmental-category value, and a health-profile value, and producing a normalized biometric value by applying a bounded-interval normalization;
   aggregating, by the one or more processors across the plurality of biometric signals, products of the contextual weight values and the normalized biometric values to produce a wellness score; and
   recording, by the one or more processors on a non-transitory storage medium, an event record linked to the composite identifier, the event record comprising the consumed-units quantity, the remaining-units quantity, the verification result, the wellness score, a timestamp of the dispensing operation, and an identifier of the medication.
42. The method of clause 41, wherein the rotatable storage member is configured to rotate about a vertical axis under control of the at least one actuator and to position the designated compartment at the dispensing position.
43. The method of clause 41, wherein the medication dispensing apparatus further comprises an output subsystem comprising at least one of a visual indicator, an audio indicator, or a haptic indicator, the method further comprising:
   defining a sequence of temporal states ordered by increasing elapsed time from the scheduled dispensing time, the sequence comprising a first temporal state extending from the scheduled dispensing time to a first time threshold, a second temporal state extending from the first time threshold to a second time threshold, and a third temporal state extending from the second time threshold to a third time threshold, each temporal state being associated with a distinct output pattern; and driving, through the communications interface, the output subsystem of the medication dispensing apparatus through the output patterns according to a current temporal state.
44. The method of clause 43, further comprising, in response to receiving a snooze response from the user, resetting the sequence to the first temporal state up to a predetermined maximum number of resets, and upon entry into the third temporal state or upon exhaustion of the predetermined maximum number of resets, transmitting an escalation notification through the communications interface to a notification recipient of a recipient hierarchy comprising a caregiver of the user, a healthcare provider of the user, and an emergency contact of the user.
45. The method of clause 41, further comprising updating the regimen-status value from the event record such that a subsequent wellness score reflects the event record, and conditioning a subsequent control instruction directed to the designated compartment on the wellness score.
46. The method of clause 41, wherein modifying the subsequent dispensing operation of the designated compartment comprises one of halting the subsequent dispensing operation until a manual override is received, delaying the subsequent dispensing operation by a configurable delay interval, or requiring an additional authorization signal from a healthcare provider device through the communications interface.
47. The method of clause 41, wherein obtaining the device identifier comprises one of scanning a quick-response code encoding the device identifier through the image sensor, receiving the device identifier through a user interface coupled to the one or more processors, or discovering the peripheral data source through a Bluetooth Low Energy interface coupled to the one or more processors.
48. A system comprising:
   one or more processors;
   one or more non-transitory storage media coupled to the one or more processors and storing a pharmacological reference data store comprising drug-interaction data and contraindication data, a reference data store of empty-container weights indexed by container category, a trained inference model trained on a corpus comprising printed prescription label images and handwritten prescription label images, a configurable tolerance value, and instructions;
   a communications interface coupled to the one or more processors;
   a wireless interface coupled to the one or more processors and configured to receive a plurality of biometric signals from a peripheral data source paired with a user; and
   a medication dispensing apparatus coupled to the communications interface and comprising a rotatable storage member carrying a plurality of compartments, a movable access member configured to expose a designated compartment of the plurality of compartments at a dispensing position, at least one actuator coupled to the movable access member and responsive to a control instruction received through the communications interface, a weight sensor configured to measure a weight of a medication container containing a medication and received in the designated compartment, an image sensor, a configuration-encoding interface configured to communicate an installed compartment count of the plurality of compartments through the communications interface, and an output subsystem, the instructions, when executed by the one or more processors, causing the system to perform operations including extracting a structured prescription record from a multi-angle image sequence of a prescription label captured by the image sensor during a continuous rotation of the medication container, deriving a composite identifier from a user profile record, a device identifier of the peripheral data source, the structured prescription record, and a placement record indicating an assignment of the medication container to the designated compartment, generating and transmitting a control instruction conditioned on a correspondence between the placement record and the designated compartment, computing a consumed-units quantity from a weight differential between a baseline container weight measurement and a current container weight measurement received from the weight sensor, generating a verification result, computing a wellness score from the plurality of biometric signals using a contextual factor vector including a regimen-status value, and recording an event record linked to the composite identifier and comprising the consumed-units quantity, a remaining-units quantity, the verification result, the wellness score, a timestamp of the dispensing operation, and an identifier of the medication.
49. The system of clause 48, wherein the plurality of compartments comprises one of four compartments, six compartments, or eight compartments.
50. The system of clause 48, wherein the rotatable storage member is configured to rotate about a vertical axis under control of the at least one actuator, and the at least one actuator comprises a servo motor having position feedback and coupled to the rotatable storage member.
51. The system of clause 48, wherein the weight sensor comprises one or more load cells positioned beneath a battery housing of the medication dispensing apparatus and configured to measure the weight of the medication container at the dispensing position with a resolution sufficient to resolve a single dose of the medication.
52. The system of clause 48, further comprising the peripheral data source, the peripheral data source comprising a wearable device worn by the user and configured to produce the plurality of biometric signals comprising at least a heart-rate signal, an activity signal, and a sleep signal.
53. The system of clause 48, wherein the trained inference model is configured to process the multi-angle image sequence and to output a plurality of prescription fields including a drug name, a dosage, a frequency, a duration, a prescribing physician, and an initial-units field.
54. One or more non-transitory computer-readable storage media storing a pharmacological reference data store comprising drug-interaction data and contraindication data, a reference data store of empty-container weights indexed by container category, a trained inference model trained on a corpus comprising printed prescription label images and handwritten prescription label images, a configurable tolerance value, and instructions that, when executed by one or more processors of a system comprising a medication dispensing apparatus having a rotatable storage member carrying a plurality of compartments, a movable access member, at least one actuator, a weight sensor, an image sensor, a configuration-encoding interface, an output subsystem, and a communications interface, cause the one or more processors to perform operations comprising:
   receiving, from the image sensor, a multi-angle image sequence of a prescription label affixed to a medication container during a continuous rotation of the medication container in a counterclockwise direction;
   applying the trained inference model to the multi-angle image sequence to extract a plurality of prescription fields including a drug name, a dosage, a frequency, a duration, a prescribing physician, and an initial-units field, and validating the plurality of prescription fields against the pharmacological reference data store to produce a structured prescription record;
   deriving a composite identifier from a user profile record of a user, a device identifier of a peripheral data source paired with the user, the structured prescription record, and a placement record indicating an assignment of the medication container to a designated compartment of the plurality of compartments;
   transmitting, through the communications interface, a control instruction addressing the designated compartment for a dispensing operation of a medication, the control instruction being conditioned on a correspondence between the placement record and the designated compartment, the control instruction causing the at least one actuator to drive the movable access member to expose the designated compartment at a dispensing position;
   computing a consumed-units quantity from a weight differential between a baseline container weight measurement and a current container weight measurement received from the weight sensor, and a per-unit-mass value associated with the medication;
   determining an initial-units quantity by selecting, in a predetermined precedence order, from the initial-units field, a quantity derived from a difference between the baseline container weight measurement and a reference empty-container weight retrieved from the reference data store using a container category of the medication container, and a default value of one hundred units;
   generating a verification result from a comparison of the consumed-units quantity to an expected-units quantity derived from the structured prescription record, and when the verification result indicates a discrepancy exceeding the configurable tolerance value, generating a subsequent control instruction modifying a subsequent dispensing operation;
   computing a wellness score from a plurality of biometric signals received from the peripheral data source using a contextual factor vector comprising a regimen-status value, an activity-level value, a symptom-severity value, a time-of-day value, a historical-deviation value, an environmental-category value, and a health-profile value combined with a set of sensitivity coefficients; and
   recording an event record linked to the composite identifier, the event record comprising the consumed-units quantity, a remaining-units quantity, the verification result, the wellness score, a timestamp of the dispensing operation, and an identifier of the medication.
55. The one or more non-transitory computer-readable storage media of clause 54, wherein the operations further comprise receiving, from the configuration-encoding interface through the communications interface, an installed compartment count of the plurality of compartments at power-on of the medication dispensing apparatus, and setting an indexing of the plurality of compartments based on the installed compartment count.
56. The one or more non-transitory computer-readable storage media of clause 54, wherein the operations further comprise:
   defining, for a scheduled dispensing time of the medication, a sequence of temporal states comprising a first temporal state extending from the scheduled dispensing time to a first time threshold, a second temporal state extending from the first time threshold to a second time threshold, and a third temporal state extending from the second time threshold to a third time threshold, each temporal state being associated with a distinct output pattern;
   driving the output subsystem through the output patterns according to a current temporal state;
   in response to receiving a snooze response from the user, resetting the sequence to the first temporal state up to a predetermined maximum number of resets; and
   upon entry into the third temporal state or upon exhaustion of the predetermined maximum number of resets, transmitting an escalation notification through the communications interface to a notification recipient of a recipient hierarchy comprising a caregiver of the user, a healthcare provider of the user, and an emergency contact of the user.
57. The one or more non-transitory computer-readable storage media of clause 54, wherein the operations further comprise updating the regimen-status value from the event record such that a subsequent wellness score reflects the event record, and conditioning the subsequent control instruction directed to the designated compartment on the wellness score.
58. The one or more non-transitory computer-readable storage media of clause 54, wherein modifying the subsequent dispensing operation comprises one of halting the subsequent dispensing operation until a manual override is received, delaying the subsequent dispensing operation by a configurable delay interval, or requiring an additional authorization signal from a healthcare provider device through the communications interface.
59. The one or more non-transitory computer-readable storage media of clause 54, wherein the operations further comprise localizing the structured prescription record into a region-appropriate format based on a jurisdictional compliance rule set stored on the one or more non-transitory computer-readable storage media prior to recording the event record.
60. The one or more non-transitory computer-readable storage media of clause 54, wherein the event record is encrypted at rest in conformance with the Health Insurance Portability and Accountability Act (HIPAA) requirements, the event record further comprises a result status of the dispensing operation indicating one of a successful dispensing, a verification failure, a missed dispensing, or a tampering event, and the operations further comprise synchronizing the event record with a cloud-based audit store at a predetermined interval.

The above described hardware description is a non-limiting example of corresponding structure for performing the functionality described herein.

Numerous modifications and variations of the present disclosure are possible in light of the above teachings. It is therefore to be understood that the invention may be practiced otherwise than as specifically described herein.

1. A method of operating a medication dispensing apparatus having a plurality of compartments, the method comprising:
obtaining, by one or more processors, a user profile record, a device identifier of a peripheral data source paired with a user, a structured prescription record for a medication associated with a medication container, and a placement record indicating an assignment of the medication container to a designated compartment of the plurality of compartments;
deriving, by the one or more processors, a composite identifier from the user profile record, the device identifier, the structured prescription record, and the placement record;
determining, by the one or more processors, whether the placement record corresponds to the designated compartment addressed for a dispensing operation of the medication;
in response to determining that the placement record corresponds to the designated compartment addressed for the dispensing operation, generating, by the one or more processors, a control instruction addressing the designated compartment;
transmitting, by the one or more processors through a communications interface to the medication dispensing apparatus, the control instruction to cause a physical state change of the designated compartment;
receiving, by the one or more processors from the medication dispensing apparatus, an outcome of the physical state change; and
recording, on a non-transitory storage medium, an event record linked to the composite identifier and indicating the outcome of the physical state change.

2. The method of claim 1, further comprising, in response to determining that the placement record does not correspond to the designated compartment addressed for the dispensing operation, inhibiting transmission of the control instruction to the medication dispensing apparatus.

3. The method of claim 2, further comprising recording, on the non-transitory storage medium, an exception event record linked to the composite identifier and indicating that transmission of the control instruction was inhibited.

4. The method of claim 1, wherein the structured prescription record is obtained by applying a trained inference model to image data of a prescription label associated with the medication container to extract a plurality of prescription fields.

5. The method of claim 4, wherein the structured prescription record is validated against a pharmacological reference data store comprising drug-interaction data or contraindication data.

6. The method of claim 4, wherein the image data comprises a multi-angle image sequence of the prescription label captured during rotation of the medication container relative to an image sensor.

7. The method of claim 1, further comprising:
receiving, from a weight sensor of the medication dispensing apparatus, a baseline container weight measurement and a current container weight measurement of the medication container;
computing a consumed-units quantity from a weight differential between the baseline container weight measurement and the current container weight measurement and a per-unit-mass value associated with the medication;
determining an initial-units quantity by selecting, in a predetermined precedence order, from (i) an initial-units field of the structured prescription record, (ii) a quantity derived from a difference between the baseline container weight measurement and a reference empty-container weight retrieved from a reference data store of empty-container weights using a container category of the medication container, and (iii) a default value; and
computing a remaining-units quantity from a difference between the initial-units quantity and the consumed-units quantity.

8. The method of claim 7, further comprising generating a verification result from a comparison of the consumed-units quantity to an expected-units quantity derived from the structured prescription record.

9. The method of claim 8, wherein a subsequent control instruction directed to the designated compartment modifies a subsequent dispensing operation of the designated compartment when the verification result indicates a discrepancy exceeding a configurable tolerance value stored on the non-transitory storage medium.

10. The method of claim 9, wherein modifying the subsequent dispensing operation comprises at least one of halting the subsequent dispensing operation, delaying the subsequent dispensing operation, or requiring an additional authorization for the subsequent dispensing operation.

11. The method of claim 1, further comprising:
receiving, from the peripheral data source, a plurality of biometric signals for the user;
for each biometric signal of the plurality of biometric signals, producing a contextual weight value by combining a contextual factor vector with a set of sensitivity coefficients, the contextual factor vector comprising a regimen-status value;
for each biometric signal of the plurality of biometric signals, producing a normalized biometric value by applying a bounded-interval normalization;
aggregating, across the plurality of biometric signals, products of the contextual weight values and the normalized biometric values to produce a wellness score; and
updating the regimen-status value from the event record such that a subsequent wellness score reflects the event record,
wherein a subsequent control instruction directed to the designated compartment is based at least in part on the wellness score.

12. The method of claim 11, wherein the contextual factor vector further comprises one or more of an activity-level value, a symptom-severity value, a time-of-day value, a historical-deviation value, an environmental-category value, or a health-profile value.

13. The method of claim 1, further comprising:
defining, for a scheduled dispensing event of the medication, a sequence of temporal states ordered by increasing elapsed time from a scheduled time, the sequence comprising at least a first temporal state, a second temporal state, and a third temporal state, each temporal state being associated with a distinct output pattern;
driving, through the communications interface, an output subsystem of the medication dispensing apparatus through the distinct output patterns according to a current temporal state;
in response to receiving a snooze response, resetting the sequence to the first temporal state a number of times bounded by a predetermined maximum; and
upon entry into the third temporal state, transmitting a notification to a notification recipient of a recipient hierarchy comprising at least one recipient distinct from the user.

14. The method of claim 1, wherein the event record is encrypted at rest on the non-transitory storage medium and is synchronized at a predetermined interval with a cloud-based audit store through the communications interface.

15. A system comprising:
one or more processors;
one or more non-transitory storage media coupled to the one or more processors and storing instructions;
a communications interface coupled to the one or more processors and configured to exchange data with a medication dispensing apparatus having a plurality of compartments; and
the medication dispensing apparatus,
wherein the medication dispensing apparatus comprises a rotatable storage member carrying the plurality of compartments, a movable access member configured to expose a designated compartment of the plurality of compartments at a dispensing position, at least one actuator configured to move the rotatable storage member, the movable access member, or both responsive to a control instruction, and a weight sensor configured to measure a weight of a medication container, and
wherein the instructions, when executed by the one or more processors, cause the system to:
obtain a user profile record, a device identifier of a peripheral data source paired with a user, a structured prescription record for a medication associated with the medication container, and a placement record indicating an assignment of the medication container to the designated compartment;
derive a composite identifier from the user profile record, the device identifier, the structured prescription record, and the placement record;
determine whether the placement record corresponds to the designated compartment addressed for a dispensing operation of the medication;
in response to determining that the placement record corresponds to the designated compartment addressed for the dispensing operation, generate the control instruction addressing the designated compartment;
transmit, through the communications interface to the medication dispensing apparatus, the control instruction to cause a physical state change of the designated compartment;
receive, from the medication dispensing apparatus, an outcome of the physical state change; and
record, on the one or more non-transitory storage media, an event record linked to the composite identifier and indicating the outcome of the physical state change.

16. The system of claim 15, wherein the instructions further cause the system to inhibit transmission of the control instruction in response to a determination that the placement record does not correspond to the designated compartment addressed for the dispensing operation.

17. The system of claim 15, wherein the medication dispensing apparatus further comprises a configuration-encoding interface configured to communicate an installed compartment count of the plurality of compartments through the communications interface, and wherein the instructions further cause the system to set an indexing of the plurality of compartments based on the installed compartment count.

18. The system of claim 15, wherein the instructions further cause the system to obtain the structured prescription record by applying a trained inference model to image data of a prescription label to extract a plurality of prescription fields, the trained inference model being trained on a corpus comprising printed prescription label images and handwritten prescription label images.

19. The system of claim 15, further comprising a wireless interface coupled to the one or more processors and configured to receive a plurality of biometric signals from the peripheral data source, wherein the instructions further cause the system to compute a wellness score from the plurality of biometric signals using a contextual factor vector comprising a regimen-status value updated from the event record, and to generate a subsequent control instruction directed to the designated compartment based at least in part on the wellness score.

20. One or more non-transitory computer-readable storage media storing instructions that, when executed by one or more processors, cause the one or more processors to perform operations comprising:
obtaining a user profile record, a device identifier of a peripheral data source paired with a user, a structured prescription record for a medication associated with a medication container, and a placement record indicating an assignment of the medication container to a designated compartment of a plurality of compartments of a medication dispensing apparatus;
deriving a composite identifier from the user profile record, the device identifier, the structured prescription record, and the placement record;
determining whether the placement record corresponds to the designated compartment addressed for a dispensing operation of the medication;
in response to determining that the placement record corresponds to the designated compartment addressed for the dispensing operation, generating a control instruction addressing the designated compartment;
transmitting the control instruction to the medication dispensing apparatus to cause a physical state change of the designated compartment;
receiving, from the medication dispensing apparatus, an outcome of the physical state change; and
recording an event record linked to the composite identifier and indicating the outcome of the physical state change.
